(19) United States
(12) Patent Application Publication
(10) Pub. No.: US 2011/0045996 A1
(43) Pub. Date: Feb. 24, 2011

(54) ROBUST REGRESSION BASED EXON ARRAY PROTOCOL SYSTEM AND APPLICATIONS

(76) Inventors: Gene Yeo, San Diego, CA (US); Fred H. Gage, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/674,436

(22) PCT Filed: Aug. 21, 2008
(86) PCT No.: PCT/US08/73934

§ 371 Date: Aug. 17, 2010

Related U.S. Application Data
(60) Provisional application No. 60/957,138, filed on Aug. 21, 2007.

(51) Int. Cl. C40B 30/02; C12Q 1/68; G01N 33/50; C12M 1/34; C40B 60/12

(52) U.S. Cl. 506/8; 435/6; 436/94; 435/287.2; 506/39

(57) ABSTRACT

An analysis technique for genetic data to detect alternative spliced exons. Exon expression of similar data is analyzed using a robust regression technique to find outliers to the main regression. False outliers are detected and removed. The remaining outliers are identified as potential alternative splicing events.

### CROSS REFERENCE TO RELATED APPLICATIONS

This application claims benefit of priority from provisional application 60/957,138 filed 21 Aug. 2007.

The above referenced documents and application and all documents referenced therein are incorporated in by reference for all purposes.

This application may be related to other patent applications and issued patents assigned to the assignee indicated above. These applications and issued patents are incorporated herein by reference to the extent allowed under applicable law.

### Precautionary Request to File an International Application, Designation of all States, and Statement that at Least One Applicant is a United States Resident or Entity

Should this document be filed electronically or in paper according to any procedure indicating an international application, Applicant hereby requests the filing of an international application and designation of all states. For purposes of this international filing, all inventors listed on a cover page or any other document filed herewith are applicants for purposes of United States National Stage filing. For purposes of this international filing, any assignees listed on a cover page or any other document filed herewith are applicants for purposes of non-United States national stage filing, or, if no assignee is listed, all inventors listed are applicants for purposes of non-United States national stage filing. For purposes of any international filing, applicants state that at least one applicant is a United States resident or United States institution. Should this application be filed in as a national application in the United States, this paragraph shall be disregarded.

### COPYRIGHT NOTICE

Pursuant to 37 C.F.R. 1.71(e), applicant notes that a portion of this disclosure contains material that is subject to and for which is claimed copyright protection (such as, but not limited to, source code listings, screen shots, user interfaces, or user instructions, or any other aspects of this submission for which copyright protection is or may be available in any jurisdiction). The copyright owner has no objection to the facsimile reproduction by anyone of the patent document or patent disclosure, as it appears in the Patent and Trademark Office patent file or records. All other rights are reserved, and all other reproduction, distribution, creation of derivative works based on the contents, public display, and public performance of the application or any part thereof are prohibited by applicable copyright law.

### APPENDIX

This application is being filed with an appendices listed as TABLE 3. These appendices and all other documents filed herewith, including documents filed in any attached Information Disclosure Statement (IDS), are incorporated herein by reference. The appendix contains further examples and information related to various embodiments of the invention at various stages of development. In particular, the appendix sets out selected source code extracts from a copyrighted software program, owned by the assignee of this patent document, which provides examples according to specific embodiments of the invention. Permission is granted to make copies of the appendices solely in connection with the making of facsimile copies of this patent document in accordance with applicable law; all other rights are reserved, and all other reproduction, distribution, creation of derivative works based on the contents, public display, and public performance of the appendix or any part thereof are prohibited by the copyright laws.

### FIELD OF THE INVENTION

The present invention relates to biological data, biological data analysis, diagnostic exons, and diagnostic sequences.

### BACKGROUND OF THE INVENTION

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

The human central nervous system is formed of many different subtypes of cells. Many of these subtypes originate from neural stem cells that migrate from a developing neural tube. The complexity of the neurons may depend on molecular, genetic and epigenetic mechanisms. Analysis of the processes that generate this diversity is used for biomedical and other research. Human embryonic stem cells are pluripotent cells that can propagate as undifferentiated cells, but can also differentiate into a multitude of cell types. Human embryonic stem cells can theoretically generate all cell types that form in an organism, and hence may form an important model for understanding human embryonic development. Embryonic stem cells can be used for generating specialized cells. One such cell line that can be formed is the neural progenitors. Both neural stem cells and progenitor cells are present throughout human development, and persist into adulthood. Different patterns within these cells have been analyzed for various purposes. For example, some studies have explored expression patterns within neural progenitor cells. Studies thus far have mostly relied on transcriptional differences between the cells.

Recent studies have suggested that up to 75% of human genes undergo alternative RNA splicing. Global analysis so far of such alternative RNA splicing has focused on comparisons across differentiated human tissues.

The Affymetrix exon array (see, for example, information on the worldwide web at affymetrix(.)com(/) products(/) arrays(/) exon_application.affx) provides a way to analyze expression of known and predicted exons in genomes. For example, the Affymetrix™ gene chip human exon array has about 5.4 million features used to interrogate around one million exon clusters with more than 1.4 million probe sets and an average of four probes per Exon. The Affymetrix™ exon array provides a means to capture expression data of a biological sample from every known and predicted exon in the human genome. The form of such large data sets and basic normalizations thereof is becoming well understood in the art. However, using such exon expression data to make useful determinations regarding biologic samples presents substantial challenges.

### SUMMARY

According to specific embodiments, the present invention is involved with methods and/or systems and/or devices that can be used together or independently to identify one or more post-transcriptional events from comparative exon expression data. According to specific embodiments of the invention, a method, referred to herein as REAP, is a general method that takes as input exon array data or similar exon expression data, generally from two or more biologic samples, and outputs indications or identifications of one or more alternative spliced (AS) exons between the samples predicted from the arrays. The exon identification method according to specific embodiments of the invention uses mainly robust regression combined with outlier detection techniques. Among the novel aspects of the method are outlier detection for the identification of alternative splicing.

Identification of alternative splicing (AS) is rapidly becoming important in a number of research settings and will have clinical applications to human disease conditions. Thus, the present invention in specific embodiments provides methods for detecting one or more AS events or related post-transcription events in research, diagnostic, manufacturing, and clinical settings.

In further embodiments, the invention involves several alternatively spliced exons (such as the alternative exon in the SLK gene) for use as molecular diagnostic tool for the pluripotent state of human embryonic stem cells and/or for other cells. These molecular markers are better than usual transcription or immunohistochemical methods as they are internally controlled: the difference in isoform ratios distinguish the state of the cell, rather than having to normalize to an external control such as GAPDH. Diagnostics based on these markers is less sensitive or not sensitive to issues such as filtering and/or image quality that can prove difficult in techniques such as immunohistochemistry).

In further embodiments, the invention involves identification of conserved candidate binding sites that are enriched proximal to REAP candidate exons. In particular, intronic cis-regulatory elements such as the FOX1/2 binding site GCAUG was identified as being proximal to candidate AS exons, suggesting that FOX1/2 may participate in the regulation of AS in NP and hESC. One or more of these conserved candidate binding sites may be used to locate candidate AS exons.

A technique is described that provides a regression-based exon array protocol based on robust regression analysis of signal estimates from an exon array. In a disclosed embodiment, the signal estimates can be from the Affymetrix™ exon array data. This can be used to identify alternatively spliced exons. One such technique is described that identifies and characterizes alternative RNA splicing events that distinguish pluripotent embryonic stem cells from multipotent neural progenitors. Thus, in further embodiments, the present invention may be understood in the context of methods and systems for biologic analysis using an appropriately programmed computer or other logic system. After reading this description it will become apparent to one of ordinary skill in the art how to implement the invention in alternative embodiments and applications. As such, this detailed description of the preferred and alternative embodiments should not be construed to limit the scope or breadth of the present invention.

While the present invention is described in detail with reference to data from exon expression arrays, the invention can be used to identify AS or other events of interest from any similar exon expression or presence data. Such data can be derived from RNA libraries, rev-trans DNA libraries, various sequencing studies of RNA, mRNA, etc., or other cellular analysis.

Various embodiments of the present invention provide methods and/or systems for analyzing large biologic data sets and/or identifying alternative splicing and/or post-transcription events that can be implemented on a general purpose or special purpose information handling appliance using a suitable programming language such as Java, C++, Cobol, C, Pascal, Fortran, PLI, LISP, assembly, etc., and any suitable data or formatting specifications, such as HTML, XML, dHTML, TIFF, JPEG, tab-delimited text, binary, etc. In the interest of clarity, not all features of an actual implementation are described in this specification. It will be understood that in the development of any such actual implementation (as in any software development project), numerous implementation-specific decisions must be made to achieve the developers' specific goals and subgoals, such as compliance with system-related and/or business-related constraints, which will vary from one implementation to another. Moreover, it will be appreciated that such a development effort might be complex and time-consuming, but would nevertheless be a routine undertaking of software engineering for those of ordinary skill having the benefit of this disclosure.

The invention and various specific aspects and embodiments will be better understood with reference to the following drawings and detailed descriptions. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that logic systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

All references, publications, patents, and patent applications cited herein are hereby incorporated by reference in their entirety for all purposes. The applicant has no intention to give to the public any disclosed embodiment. Among the disclosed changes and modifications, those which may not literally fall within the scope of the patent claims constitute, therefore, a part of the present invention in the sense of doctrine of equivalents.

A description of experiments and methods related to the present invention is included in: Yeo G W, Xu X, Liang T Y, Muotri A R, Carson C T, et al. (2007) Alternative splicing events identified in human embryonic stem cells and neural progenitors. PloSComput Biol 3(10): e196. doi:10(.)1371/journal(.)pcbi.0030196, which is incorporated herein by reference including all supporting tables and figures. Various example exon expression data has been made available at www(.)snl.salk.edu(/)˜geneyeo/stuff/papers/supplementary/ES-NP/ consisting of the files:

| | |
|---|---|
| GY06091401.CEL | hCNS-SCns |
| GY06091402.CEL | hCNS-SCns |
| GY06091403.CEL | hCNS-SCns |
| GY060914A.CEL | Cyt-ES |
| GY060914B.CEL | Cyt-ES |
| GY060914C.CEL | Cyt-ES |
| GY061115HFB1.CEL | fetal brain |
| GY061115HFB2.CEL | fetal brain |
| GY070109hnpa.CEL | Cyt-NP |
| GY070109hnpb.CEL | Cyt-NP |
| GY070109hnpc.CEL | Cyt-NP |
| GY070109hues6a.CEL | HUES6-ES |
| GY070109hues6b.CEL | HUES6-ES |
| GY070109hues6c.CEL | HUES6-ES |
| GY070220Hues6NPa.CEL | HUES6-NP |
| GY070220Hues6NPb.CEL | HUES6-NP |
| GY070220Hues6NPc.CEL | HUES6-NP |

### REFERENCES

The following references provide various background and other information to provide a context for understanding aspects of the invention. These references are incorporated herein by reference for all purposes.
1. Muotri A R, Chu V T, Marchetto M C, Deng W, Moran J V, et al. (2005) Somatic mosaicism in neuronal precursor cells mediated by L1 retrotransposition. Nature 435: 903-910.
2. Muotri A R, Gage F H (2006) Generation of neuronal variability and complexity. Nature 441: 1087-1093.
3. Thomson J A, Itskovitz-Eldor J, Shapiro S S, Waknitz M A, Swiergiel J J, et al. (1998) Embryonic stem cell lines derived from human blastocysts. Science 282: 1145-1147.
4. Keller G (2005) Embryonic stem cell differentiation: emergence of a new era in biology and medicine. Genes Dev 19: 1129-1155.
5. Sonntag K C, Simantov R. Isacson O (2005) Stem cells may reshape the prospect of Parkinson's disease therapy. Brain Res Mol Brain Res 134: 34-51.
6. Reubinoff B E, Itsykson P, Turetsky T, Pera M F, Reinhartz E, et al. (2001) Neural progenitors from human embryonic stem cells. Nat Biotechnol 19: 1134-1140.
7. Carpenter M K, Inokuma M S, Denhatn J, Mujtaba T, Chiu C P, et al. (2001) Enrichment of neurons and neural precursors from human embryonic stein cells. Exp Neurol 172: 383-397.
8. Perrier A L, Tabar V, Barberi T, Rubio M E, Bruses J, et al. (2004) Derivation of midbrain dopamine neurons from human embryonic stein cells. Proc Natl Acad Sci USA 101: 12543-12548.
9. Li X J, Du Z W, Zarnowska E D, Pankratz M, Hansen L O, et al. (2005) Specification of motoneurons from human embryonic stem cells. Nat Biotechnol 23: 215-221.
10. Yan Y, Yang D, Zarnowska E D, Du Z, Werbel B, et al. (2005) Directed differentiation of dopaminergic neuronal subtypes from human embryonic stem cells. Stem Cells 23: 781-790.
11. Nistor G I, Totoiu M O, Hague N, Carpenter M K, Keirstead H S (2005) Human embryonic stein cells differentiate into oligodendrocytes in high purity and myelinate after spinal cord transplantation. Glia 49: 385-396.
12. Muotri A R, Nakashima K, Toni N, Sandler V M, Gage F H (2005) Development of functional human embryonic stem cell-derived neurons in mouse brain. Proc Natl Acad Sci USA 102: 18644-18648.
13. Cai J, Chen J, Liu Y, Miura T, Luo Y, et al. (2006) Assessing self-renewal and differentiation in human embryonic stem cell lines. Stem Cells 24: 516-530.
14. Bhattacharya B, Cai J, Luo Y, Miura T, Mejido J, et al. (2005) Comparison of the gene expression profile of undifferentiated human embryonic stem cell lines and differentiating embryoid bodies. BMCDev Biol 5: 22.
15. Miura T, Luo Y, Khrebtukova I, Brandenberger R, Zhou D, et al. (2004) Monitoring early differentiation events in human embryonic stem cells by massively parallel signature sequencing and expressed sequence tag scan. Stem Cells Dev 13: 694-715.
16. Brandenberger R, Wei H, Zhang S, Lei S, Murage J, et al. (2004) Transcriptome characterization elucidates signaling networks that control human ES cell growth and differentiation. Nat Biotechnol 22: 707-716.
17. Brandenberger R, Khrebtukova I, Thies R S, Miura T, Jingli C, et al. (2004) MPSS profiling of human embryonic stem cells. BMCDev Biol 4: 10.
18. Gage F H, Ray J, Fisher L J (1995) Isolation, characterization, and use of stem cells from the CNS. Annu Rev Neurosci 18: 159-192.
19. Weiss S, Dunne C, Hewson J, Wohl C, Wheatley M, et al. (1996) Multipotent CNS stem cells are present in the adult mammalian spinal cord and ventricular neuroaxis. J Neurosci 16: 7599-7609.
20. Weissman I L (2000) Stem cells: units of development, units of regeneration, and units in evolution. Cell 100: 157-168.
21. Taylor H, Minger S L (2005) Regenerative medicine in Parkinson's disease: generation of mesencephalic dopaminergic cells from embryonic stem cells. Curr Opin Biotechnol 16: 487-492.
22. Hermann A, Gerlach M, Schwarz J, Storch A (2004) Neurorestoration in Parkinson's disease by cell replacement and endogenous regeneration. Expert Opin Biol Ther 4: 131-143.
23. Uchida N, Buck D W, He D, Reitsma M J, Masek M, et al. (2000) Direct isolation of human central nervous system stem cells. Proc Natl. Acad Sci USA 97: 14720-14725.
24. Wright L S, Li J, Caldwell M A, Wallace K, Johnson J A, et al. (2003) Gene expression in human neural stem cells: effects of leukemia inhibitory factor. J Neurochem 86: 179-195.
25. Storch A, Paul G, Csete M, Boehm B O, Carvey P M, et al. (2001) Long-term proliferation and dopaminergic differentiation of human mesencephalic neural precursor cells. Exp Neurol 170: 317-325.
26. Arsenijevic Y, Villemure J G, Brunet J F, Bloch J J, Deglon N, et al. (2001) Isolation of multipotent neural precursors residing in the cortex of the adult human brain. Exp Neurol 170: 48-62.
27. Cai J, Shin S, Wright L, Liu Y, Zhou D, et al. (2006) Massively parallel signature sequencing profiling of fetal human neural precursor cells. Stem Cells Dev 15: 232-244.
28. Nunes M C, Roy N S, Keyoung H M, Goodman R R, McKhann G Jr, et al. (2003) Identification and isolation of multipotential neural progenitor cells from the subcortical white matter of the adult human brain. Nat Med 9: 439-447.
29. Moe M C, Westerlund U, Varghese M, Berg-Johnsen J, Svensson M, et al. (2005) Development of neuronal networks from single stem cells harvested from the adult human brain. Neurosurgery 56: 1182-1188; discussion 1188-1190.
30. Kukekov V G, Laywell E D, Suslov O, Davies K, Scheffler B, et al. (1999) Multipotent stem/progenitor cells with similar properties arise from two neurogenic regions of adult human brain. Exp Neurol 156: 333-344.
31. Kirschenbaum B, Nedergaard M, Preuss A, Barami K, Fraser R A, et al. (1994) In vitro neuronal production and differentiation by precursor cells derived from the adult human forebrain. Cereb Cortex 4: 576-589.
32. Johansson C B, Momma S, Clarke D L. Risling M, Lendahl U, et al. (1999) Identification of a neural stem cell in the adult mammalian central nervous system. Cell 96: 25-34.
33. Hermann A, Maisel M, Liebau S, Gerlach M, Kleger A, et al. (2006) Mesodermal cell types induce neurogenesis from adult human hippocampal progenitor cells. J Neurochem 98: 629-640.
34. Westerlund U, Moe M C, Varghese M. Berg-Johnsen J, Ohlsson M. et al. (2003) Stem cells from the adult human brain develop into functional neurons in culture. Exp Cell Res 289: 378-383.
35. Maisel M, Herr A, Milosevic J, Hermann A. Habisch H J, et al. (2007) Transcription profiling of adult and fetal human neuroprogenitors identifies divergent paths to maintain the neuroprogenitor cell state. Stem Cells 25: 224-234.
36. Black D L (2003) Mechanisms of alternative pre-messenger RNA splicing. Annu Rev Biochem 72: 291-336.
37. Cartegni L, Chew S L, Krainer A R (2002) Listening to silence and understanding nonsense: exonic mutations that affect splicing. Nat Rev Genet 3: 285-298.
38. Graveley B R (2001) Alternative splicing: increasing diversity in the proteomic world. Trends Genet 17: 100-107.
39. Zavolan M, Kondo S, Schonbach C, Adachi J, Hume D A, et al. (2003) Impact of alternative initiation, splicing, and termination on the diversity of the mRNA transcripts encoded by the mouse transcriptome. Genome Res 13: 1290-1300.
40. Blencowe B J (2006) Alternative splicing: new insights from global analyses. Cell 126: 37-47.
41. Black D L, Grabowski P J (2003) Alternative pre-mRNA splicing and neuronal function. Prog Mol Subcell Biol 31: 187-216.
42. Grabowsld P J, Black D L (2001) Alternative RNA splicing in the nervous system. Prog Neurobiol 65: 289-308.
43. Ule J, Jensen K B, Ruggiu M, Mele A, Ule A, et al. (2003) CLIP identifies Nova-regulated RNA networks in the brain. Science 302: 1212-1215.
44. Jensen K B, Dredge B K, Stefani G, Thong R, Buckanovich R J, et al. (2000) Nova-1 regulates neuron-specific alternative splicing and is essential for neuronal viability. Neuron 25: 359-371.
45. Rahman L, Bliskovski. V, Reinhold W, Zajac-Kaye M (2002) Alternative splicing of brain-specific PTB defines a tissue-specific isoform pattern that predicts distinct functional roles. Genomics 80: 245-249.
46. Ashiya M, Grabowski P J (1997) A neuron-specific splicing switch mediated by an array of pre-mRNA repressor sites: evidence of a regulatory role for the polypyrimidine tract binding protein and a brain-specific PTB counterpart. Rna 3: 996-1015.
47. Boutz P L, Stoilov P, Li Q, Lin C H, Chawla G, et al. (2007) A posttranscriptional regulatory switch in polypyrimidine tract-binding proteins reprograms alternative splicing in developing neurons. Genes Dev 21: 1636-1652.
48. Krawczak M, Reiss J, Cooper D N (1992) The mutational spectrum of single base-pair substitutions in mRNA splice junctions of human genes: causes and consequences. Hum Genet 90: 41-54.
49. Faustino N A, Cooper T A (2003) Pre-mRNA splicing and human disease. Genes Dev 17: 419-437.
50. Yeo G, Holste D, Kreiman G, Burge C B (2004) Variation in alternative splicing across human tissues. Genome Biol 5: R74.
51. Xu Q, Modrek B, Lee C (2002) Genome-wide detection of tissue-specific alternative splicing in the human transcriptome. Nucleic Acids Res 30: 3754-3766.
52. Johnson J M, Castle J, Garrett-Engele P, Kan Z. Loerch P M, et al. (2003) Genome-wide survey of human alternative pre-mRNA splicing with exon junction microarrays. Science 302: 2141-2144.
53. Pritsker M, Doniger T T, Kramer L C, Westcot S E, Lemischka I R (2005) Diversification of stem cell molecular repertoire by alternative splicing. Proc Natl Acad Sci USA 102: 14290-14295.
54. Abeyta M J, Clark A T, Rodriguez R T, Bodnar M S, Pera R A, et al. (2004) Unique gene expression signatures of independently-derived human embryonic stem cell lines. Hum Mol Genet 13: 601-608.
55. Yeo G W, Van Nostrand E, Holste D, Poggio T, Burge C B (2005) Identification and analysis of alternative splicing events conserved in human and mouse. Proc Natl Acad Sci USA 102: 2850-2855.
56. Cowan C A, Klimanskaya I, McMahon J. Atienza J. Witmyer J, et al. (2004) Derivation of embryonic stem-cell lines from human blastocysts. N Engl J Med 350: 1353-1356.
57. Lowell S, Benchoua A, Heavey B, Smith AG (2006) Notch promotes neural lineage entry by pluripotent embryonic stem cells. PLoS Biol 4: e121. doi:10.1371/journal.pbio.0040121
58. Androutsellis-Theotokis A, Leker R R. Soldner F, Hoeppner D J, Ravin R, et al. (2006) Notch signalling regulates stem cell numbers in vitro and in vivo. Nature 442: 823-826.
59. Eiraku M, Tohgo A, Ono K, Kaneko M, Fujishima K, et al. (2005) DNER acts as a neuron-specific Notch ligand during Bergmann glial development. Nat Neurosci 8: 873-880.
60. Pevny L H, Sockanathan S, Placzek M, Lovell-Badge R (1998) A role for SOX1 in neural determination. Development 125: 1967-1978.
61. Baldassarre G, Romano A, Annenante F, Rambaldi M, Paoletti I, et al. (1997) Expression of teratocarcinoma-derived growth factor-1 (TDGF-1) in testis germ cell tumors and its effects on growth and differentiation of embryonal carcinoma cell line NTERA2/D1. Oncogene 15: 927-936.
62. Xu C, Liguori G, Adamson E D, Persico M G (1998) Specific arrest of cardiogenesis in cultured embryonic stein cells lacking Cripto-1. Dev Biol 196: 237-247.
63. Pesce M, Scholer H R (2001) Oct.-4: gatekeeper in the beginnings of mammalian development. Stem Cells 19: 271-278.
64. Mitsui K, Tokuzawa Y, Itoh H, Segawa K, Murakami M, et al. (2003) The homeoprotein Nanog is required for maintenance of pluripotency in mouse epiblast and ES cells. Cell 113: 631-642.
65. Zhang J Z, Gao W, Yang H B, Zhang B, Zhu Z Y, et al. (2006) Screening for genes essential for mouse embryonic stem cell self-renewal using a subtractive RNA interference library. Stem Cells 24: 2661-2668.
66. Yeo G W, Nostrand E L, Liang T Y (2007) Discovery and analysis of evolutionarily conserved intronic splicing regulatory elements. PLoS Genet 3: e85. doi:10.1371/journal.pgen.0030085 67. Gorlach M, Burd C O. Dreyfuss G (1994) The determinants of RNA-binding specificity of the heterogeneous nuclear ribonucleoprotein C proteins. J Biol Chem 269: 23074-23078.
68. Faustino N A, Cooper T A (2005) Identification of putative new splicing targets for ETR-3 using sequences identified by systematic evolution of ligands by exponential enrichment. Mol Cell Biol 25: 879-887.
69. Chan R C, Black D L (1997) Conserved intron elements repress splicing of a neuron-specific c-src exon in vitro. Mol Cell Biol 17: 2970.
70. Huh G S, Hynes R O (1994) Regulation of alternative pre-mRNA splicing by a novel repeated hexanucleotide element. Genes Dev 8: 1561-1574.
71. Hedjran F, Yeakley J M, Huh G S, Hynes R O, Rosenfeld M G (1997) Control of alternative pre-mRNA splicing by distributed pentameric repeats. Proc Natl Acad Sci USA 94: 12343-12347.
72. Lim L P, Sharp P A (1998) Alternative splicing of the fibronectin HUB exon depends on specific TGCATG repeats. Mol Cell Biol 18: 3900-3906.
73. Underwood J G, Boutz P L, Dougherty J D, Stoilov P, Black D L (2005) Homologues of the Caenorhabditis elegans Fox-1 protein are neuronal splicing regulators in mammals. Mol Cell Biol 25: 10005-10016.
74. Dredge B K, Darnell R B (2003) Nova regulates GABA(A) receptor gamma2 alternative splicing via a distal downstream UCAU-rich intronic splicing enhancer. Mol Cell Biol 23: 4687-4700.
75. Han K, Yeo G, An P, Burge C B, Grabowski P J (2005) A combinatorial code for splicing silencing: UAGG and GGGG motifs. PLoS Biol 3: e158. doi:10.1371/journal.pbio.0030158
76. Wu H, Xu J, Pang Z P, Ge W, Kim K J, et al. (2007) integrative genomic and functional analyses reveal neuronal subtype differentiation bias in human embryonic stem cell lines. Proc Natl Acad Sci USA 104: 13821-13826.
77. Sugnet C W, Kent W J, Ares M Jr, Haussler D (2004) Transcriptome and genome conservation of alternative splicing events in humans and mice. Pac Symp Biocomput 2004: 66-77.
78. Sorek R, Ast G (2003) Intronic sequences flanking alternatively spliced exons are conserved between human and mouse. Genome Res 13: 1631-1637.
79. Zhang Y H, Hume K, Cadonic R, Thompson C, Hakim A, et al. (2002) Expression of the Ste20-like kinase SLK during embryonic development and in the murine adult central nervous system. Brain Res Dev Brain Res 139: 205-215.
80. Karolchik D, Baertsch R, Diekhans M, Furey T S, Hinrichs A, et al. (2003) The UCSC Genome Browser Database. Nucleic Acids Res 31: 51-54.
81. Belsley D A, Kuh E, Welsch R E (1980) Regression diagnostics: identifying influential data and sources of collinearity. New York: John Wiley and Sons.

### BRIEF DESCRIPTION OF THE DRAWINGS

FIG. 1 illustrates a basic flowchart of a method for identifying AS events according to specific embodiments of the invention.

FIG. 2 is a block diagram showing a representative example logic device in which various aspects of the present invention may be embodied.

FIG. 3A-F illustrate a REAP method comparing exon array signal estimates from hCNS-SCns and Cyt-ES according to specific embodiments of the invention.

FIG. 4A-C show sources and detection of false positives.

FIG. 5A-C show (B) Nine RT-PCR validated REAP[+] AS events in hESCs (Cyt-ES and HUES6-ES), derived NPs (Cyt-NP and HUES6-NP), and hCNS-SCns. Arrows indicate the larger (exon-included) isoforms and smaller (exon-skipped) isoforms. The nine are labeled EHBP1, SLK, RAI14, CTTN, SORBS1, UNC84A, SIRT1, MLLT10, POT1.

FIG. 6 illustrates a Correlation between “Outliers” according to specific embodiments of the invention. (A) The number of probesets with N significant “outliers” was determined for hCNS-SCns versus Cyt-ES, hCNS-SCns versus HUES6-ES, Cyt-NPs versus Cyt-ES, and HUES6-NPs versus HUES6-ES (N=0, 1, 2, 3, 4, 5). For comparison, points to probeset relationships were randomly permuted, retaining the same number of “outliers.” Vertical bars represent the ratio between the number of actual points and the randomly permutated sets. (B) Similar to (A), except points were counted as “outliers” only if they were “outliers” in both hCNS-SCns versus Cyt-ES and hCNS-SCns versus HUES6-ES (combined hCNS-SCns versus hESC; blue bars); in both HUES6-NP versus HUES6-ES and Cyt-NP versus Cyt-ES (combined derived NP versus hESC; red bars); and in all four comparisons (combined NP versus hESC; yellow bar).

Table 1 lists DNA base sequences that may be predictive of AS regions according to specific embodiments of the invention. The table lists conserved 5-mers enriched in Downstream(DO) or Upstream(UP) Intronic Regions of REAPH Exons Included in ES (NP) and Skipped in NP (ES). For example, in row 6 ACCTG was enriched in the downstream intronic regions of exons included in ES and skipped in NP, relative to REAP[−] exons.

Table 2 lists alternative splice exons for detection of stem cells according to specific embodiments of the invention.

Table 3 lists example computer program code listing for detection of candidate AS exons according to specific embodiments of the invention.

### DESCRIPTION OF SPECIFIC EMBODIMENTS

Before describing the present invention in detail, it is to be understood that this invention is not limited to particular compositions or systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content and context clearly dictates otherwise. Thus, for example, reference to “a device” includes a combination of two or more such devices, and the like. Unless defined otherwise, technical and scientific terms used herein have meanings as commonly understood by one of ordinary skill in the art to which the invention pertains. Although any methods and materials similar or equivalent to those described herein can be used in practice or for testing of the present invention, the preferred materials and methods are described herein. Unless the context requires otherwise, throughout the specification and claims which follow, the word “comprise” and variations thereof, such as, “comprises” and “comprising” are to be construed in an open, inclusive sense, that is as “including, but not limited to.” The headings provided herein are for convenience only and do not interpret the scope or meaning of the claimed invention.

### 1. Overview

The ability of embryonic stem cells to generate all three embryonic germ layers has raised the exciting possibility that human embryonic stem cells (hESCs) may become an unlimited source of cells or tissues for transplantation therapies involving organs or tissues such as the liver, pancreas, blood and nervous system and tools to explore the molecular mechanisms of human development. Despite such interest, relatively little is understood about the molecular mechanisms defining their pluripotency and the molecular changes important for hESCs to differentiate into specific cell types. To understand these events, protocols have been and are still being developed to differentiate embryonic stem cells into a variety of lineages.

Of particular biomedical interest is in the capacity of hESCs to be differentiated into a self-renewing population of neuroprogenitor cells (NPs) that can be then further coaxed into a variety of neuronal subtypes, such as dopaminergic neurons that are important in the etiology and treatment of Parkinson's disease or cholinergic neurons, important in the etiology and treatment of Amyotrophic Lateral Sclerosis (ALS). While many microarray studies have explored molecular differences between hESCs and derived NPs, most if not all have focused on transcriptional changes. These studies have largely ignored intermediate RNA processing events prior to and during translation. In recent years, alternative splicing has gained momentum as being important in normal development, apoptosis and cancer.

Human embryonic stem cells (hESCs) and neural progenitor (NP) cells are excellent models for recapitulating early neuronal development in vitro, and are key to establishing strategies for the treatment of degenerative disorders. While much effort had been undertaken to analyze transcriptional and epigenetic differences during the transition of hESC to NP, very little work has been performed to understand post-transcriptional changes during neuronal differentiation. Alternative splicing (AS) of RNA, a major form of post-transcriptional gene regulation, is important in mammalian development and neuronal function.

Deriving neural progenitors (NP) from human embryonic stem cells (hESC) is an important step in creating homogeneous populations of cells that will differentiate into myriad neuronal subtypes necessary to form a human brain. During RNA alternative splicing (AS), non-coding sequences (introns) in a pre-mRNA are differentially removed in different cell types and tissues, and the remaining sequences (exons) are joined to form multiple forms of mature RNA, playing an important role in cellular diversity.

AS is frequently used to regulate gene expression and to generate tissue-specific mRNA and protein isoforms [36-39]. Recent studies using splicing-sensitive microarrays suggested that up to 75% of human genes undergo AS, where multiple isoforms are derived from the same genetic loci [40]. This functional complexity underscores the challenge and importance of elucidating AS regulation. AS appears to play a dominant role in regulating neuronal gene expression and function [41,42]. Examples of splicing regulators that are enriched and function specifically in neuronal cells include the brain-specific splicing factor Nova [43,44] and neural-specific polypyrimidine tract binding protein (nPTB), which antagonizes its paralogous PTB to regulate exon exclusion in neuronal cells [45-47]. Finally, an early report estimating that 15% of point mutations disrupt splicing underscores the importance of splicing in human disease [48]. Indeed, the disruption of specific AS events has been implicated in several human genetic diseases, such as frontotemporal dementia and parkinsonism, Frasier syndrome, and atypical cystic fibrosis [49]. Insights into the regulation of AS have come predominantly from the molecular dissection of individual genes [36,49].

Most systematic global analyses on AS have focused on comparisons across differentiated human tissues [50-52]. Only one study, utilizing expressed sequence tag (EST) collections from stem cells, has attempted to find AS differences between embryonic and hematopoietic stem cells [53]. However, utilizing ESTs to identify AS has intrinsic problems, as ESTs tend to be biased for the 39 ends of genes, and full coverage of the genome by ESTs is severely limited by sequencing costs.

According to specific embodiments of the invention, the present invention is directed to systems and methods for identifying AS events and/or related post-transcriptional events, using exon analysis. The invention has applications to identifying AS exons for individual genes as well as for analyzing large exon expression data sets. Affymetrix™ exon arrays provide an approach to interrogate the expression of every known and predicted exon in the human genome and generate the large exon expression data sets analyzed by embodiments of the current invention. As an example, the Affymetrix GeneChip Human Exon 1.0 ST array contains 5.4 million features used to interrogate 1 million exon clusters (collections of overlapping) of known and predicted exons with more than 1.4 million probesets, with an average of four probes per exon. Particular embodiments are directed to identifying AS events that distinguish pluripotent hESCs from multipotent NPs, paving the way for future candidate gene approaches to study the impact of AS in hESCs and NPs.

According to specific embodiments of the invention, data from exon arrays with probes targeting hundreds of thousands of exons is analyzed using a novel Robust-Regression-based Exon Array Protocol (REAP) computational method. REAP AS candidates have been shown as consistent with other types of methods for discovering alternative exons.

According to specific embodiments of the invention, REAP was used to study AS comparing human ES to NP. According to specific embodiments of the invention, REAP predictions have been found to be enriched in genes encoding serine/threonine kinase and helicase activities. An example is a REAP-predicted alternative exon in the SLK (serine/threonine kinase 2) gene that is differentially included in hESC, but skipped in NP as well as in other differentiated tissues. Lastly, comparative sequence analysis revealed conserved intronic cis-regulatory elements such as the FOX1/2 binding site GCAUG as being proximal to candidate AS exons, suggesting that FOX1/2 may participate in the regulation of AS in NP and hESC. By comparing genomic sequences across multiple mammals, methods according to specific embodiments of the invention identified dozens of conserved candidate binding sites that were enriched proximal to REAP candidate exons.

In further specific example implementations and experiments, the invention was applied to discover distinguishing alternative splicing events in hESCs, their derived NPs, and hCNS-SCns. REAP predictions in this case were found to correlate well with transcript-based methods for identifying alternative exons. Interestingly, this finding suggested that current databases of transcript information, albeit not specifically enriched for embryonic or neural progenitors, in aggregate are nevertheless predictive of alternative splicing events.

According to specific embodiments of the invention, various cell types (e.g., hESCs, NP derived from hESC, and human central nervous system stem cells (hCNS-SC)) were compared using Affymetrix exon arrays. REAP outlier detection in one set of example experiments identified 1,737 internal exons that are predicted to undergo AS in NP compared to hESC. Experimental validation of REAP-predicted AS events indicated a threshold-dependent sensitivity ranging from 56% to 69%, at a specificity of 77% to 96%. REAP predictions significantly overlapped sets of alternative events identified using expressed sequence tags (ESTs) and evolutionarily conserved AS events. Results also reveal that focusing on differentially expressed genes between hESC and NP will overlook 14% of potential AS genes.

In a particular example experiment, because different hESC lines were established under different culture conditions from embryos with unique genetic backgrounds, it was expected that hESCs and their derived NPs might have distinct epigenetic and molecular signatures [54]. As both common and cell-line specific alternatively spliced exons are likely to be important in regenerative research, in these experiments two separate hESC lines were used, with independent protocols for differentiating the hESCs into NPs positive for Sox1, an early neuroectodermal marker. As an endogenously occurring population of NPs, human central nervous system stem cells grown as neurospheres (hCNS-SCns) were utilized as a natural benchmark for derived NPs.

In one example application of the invention, RNA from two cell populations, embryonic stem cells and neural progenitor cells was extracted and processed and hybridized on to Affymetrix™ exon arrays. While Affymetrix™ exon arrays are described in the embodiments, other embodiments may use other kinds of array readouts or systems useful for deriving similar data. As previously noted, however, the invention is applicable to any type of exon expression or presence data, however derived.

Independent protocols were used for differentiating the stem cells into neural progenitors that are positive for Sox1, an early neuroectodermal marker. In the specific experiment, neuroprogenitor cells (Cyt-NP, for example, or HUES6-NP) were derived from embryonic cells (ES, for example, Cyt-ES and HUES6-NP, respectively). An embodiment uses human central nervous system stem cells grown as neurospheres as a natural benchmark against which comparisons can be made.

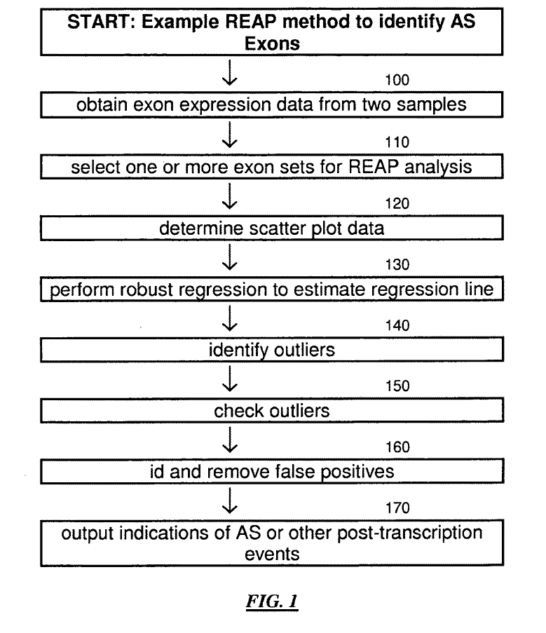

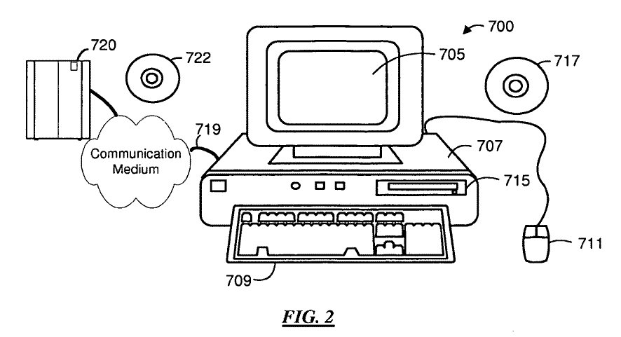

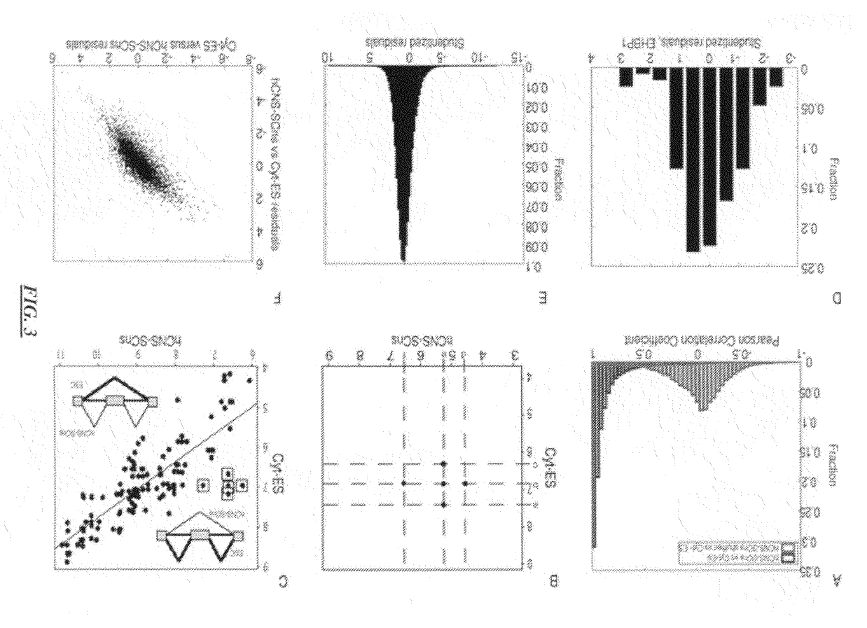

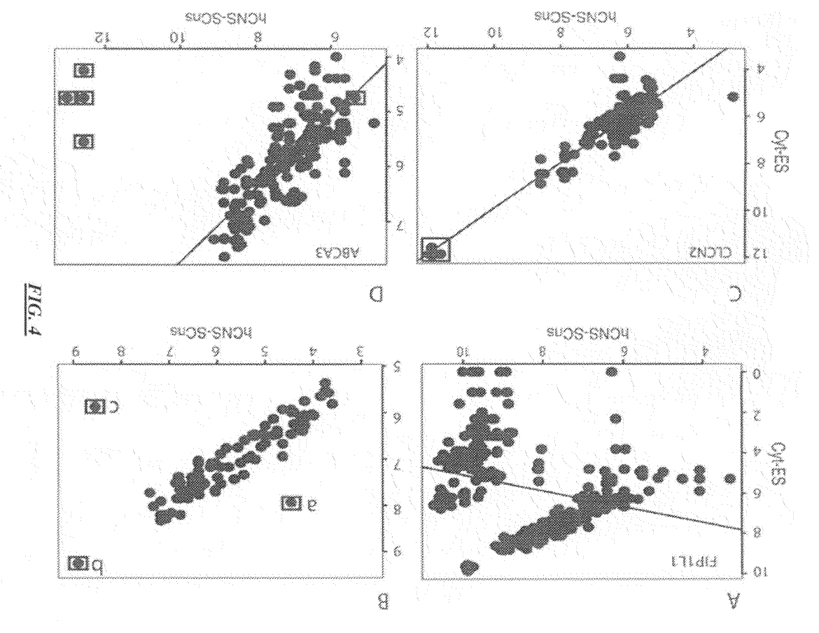

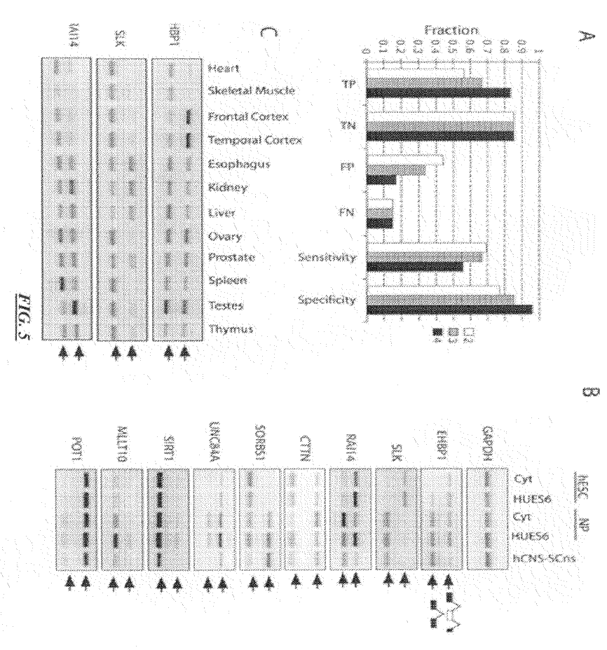

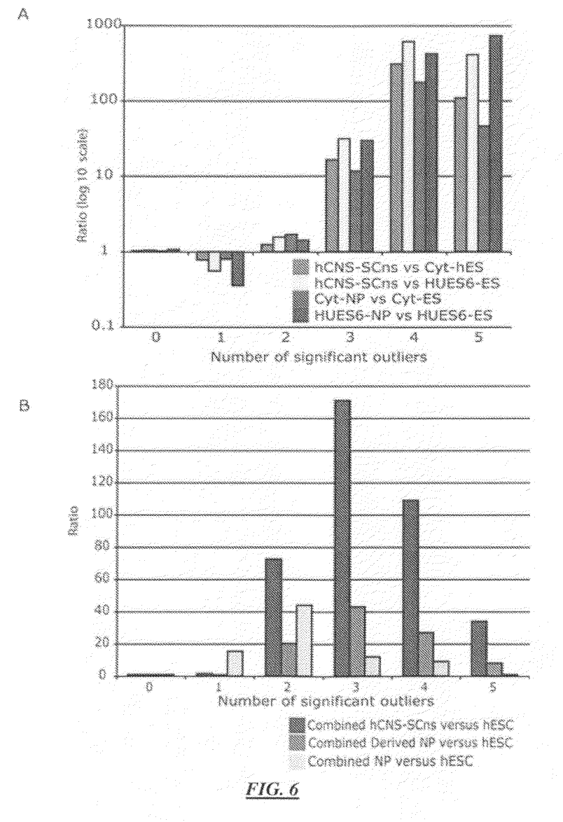

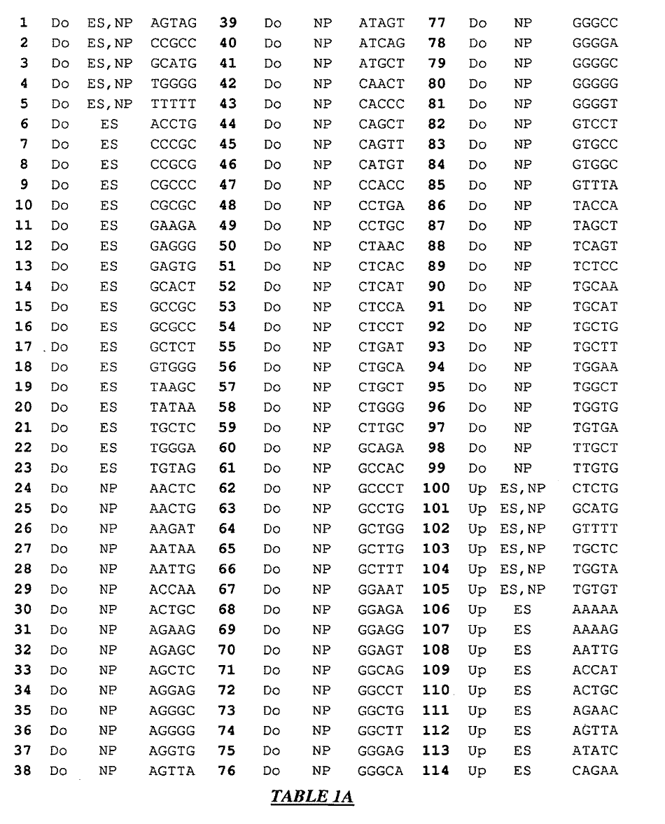

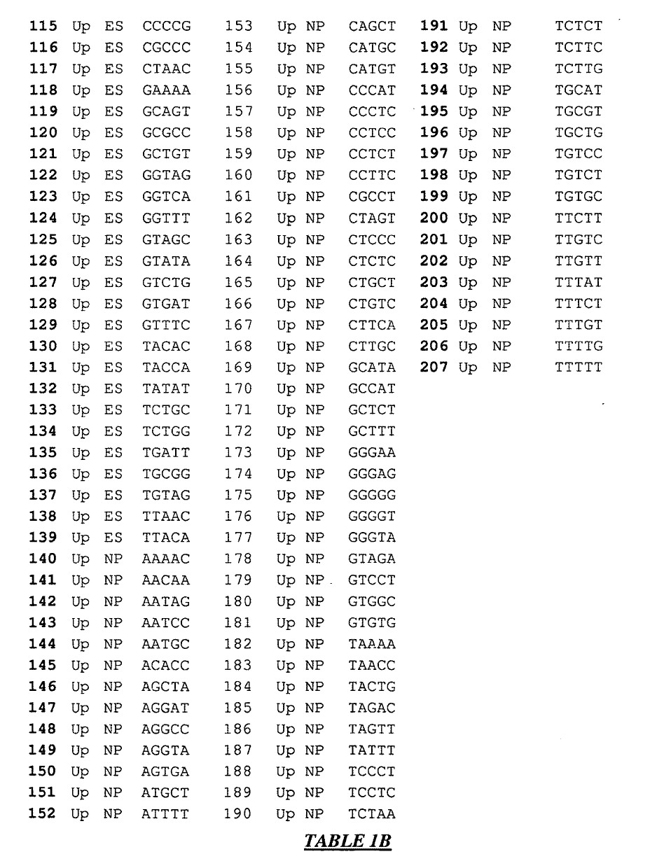

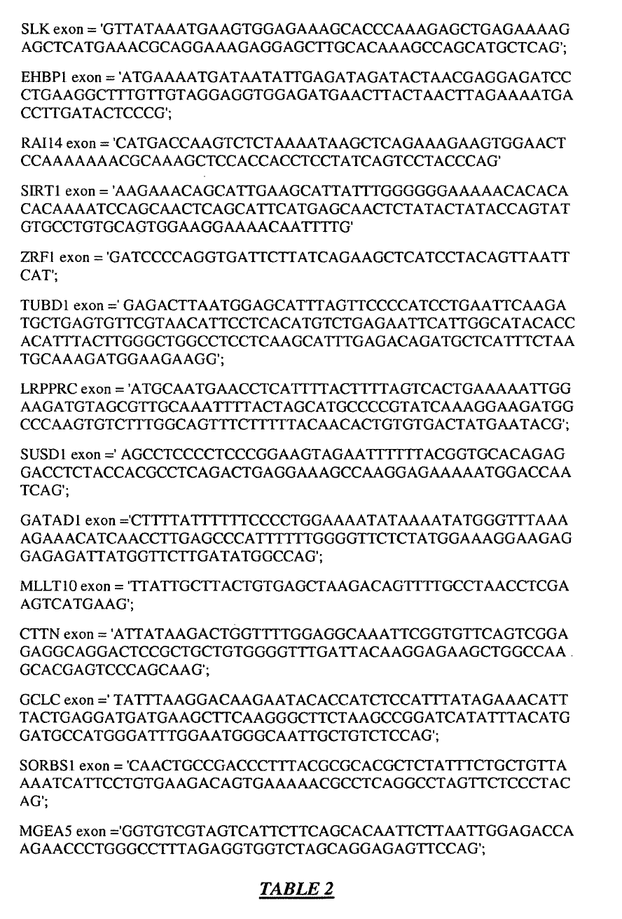

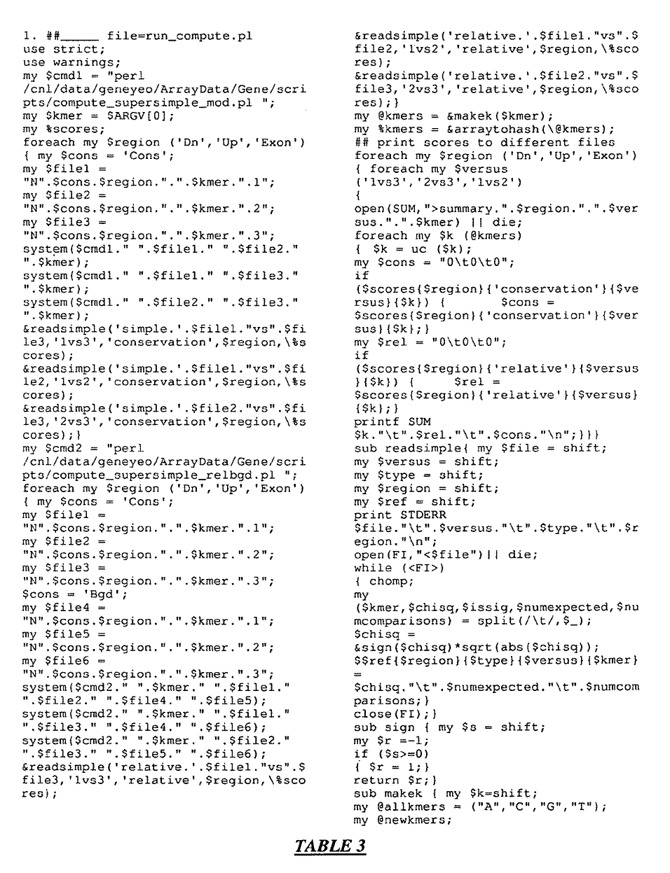

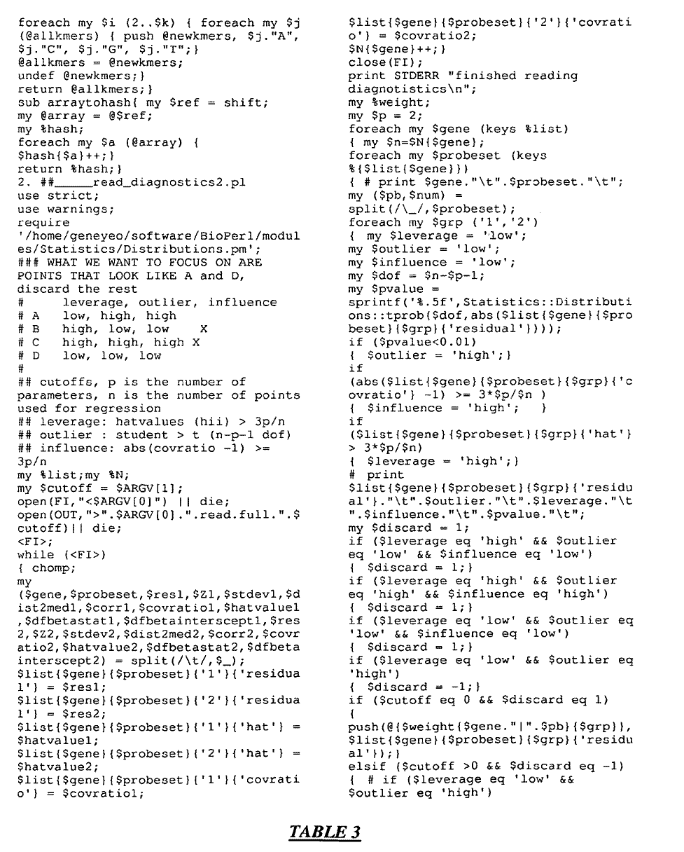

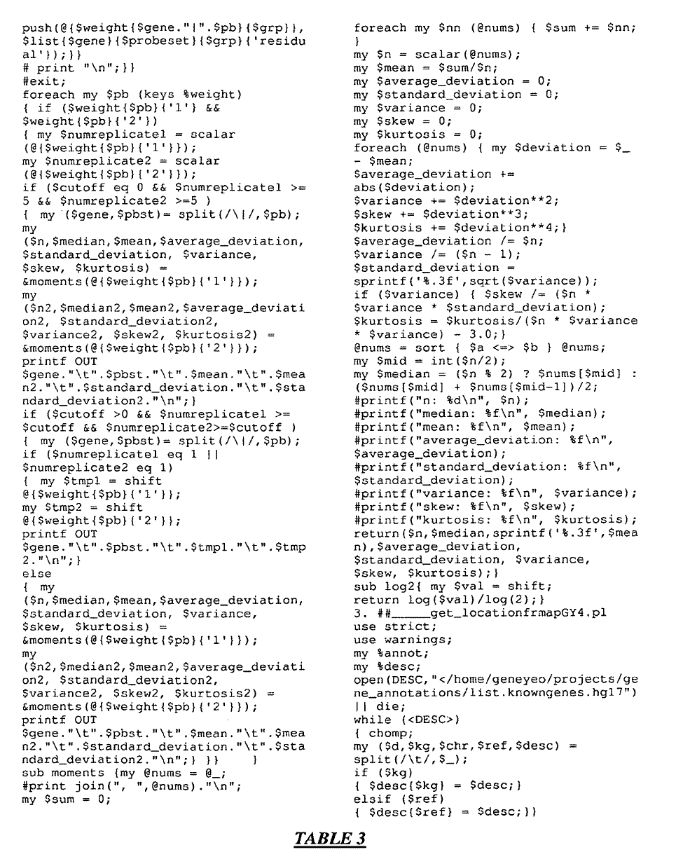

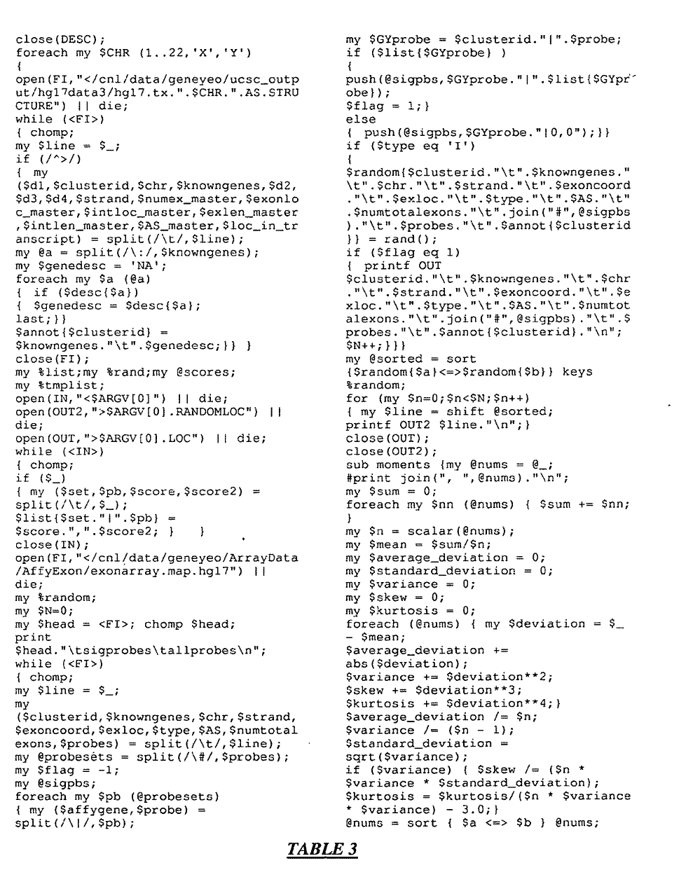

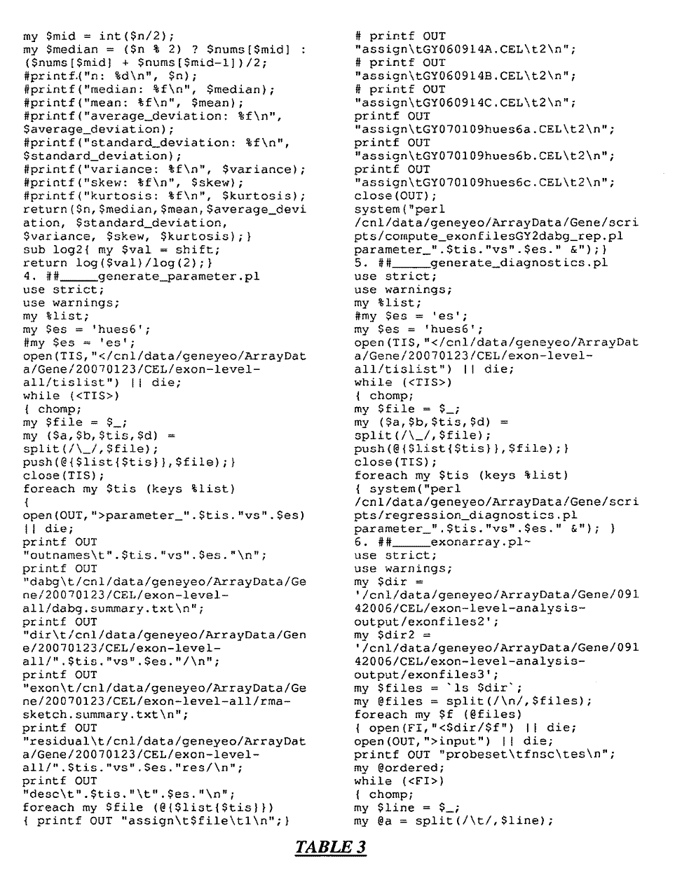

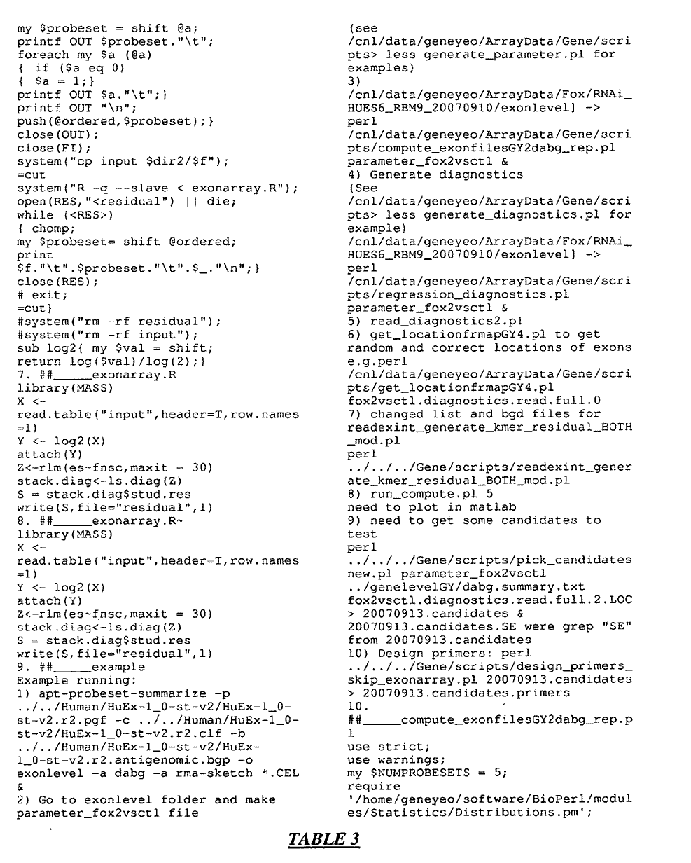

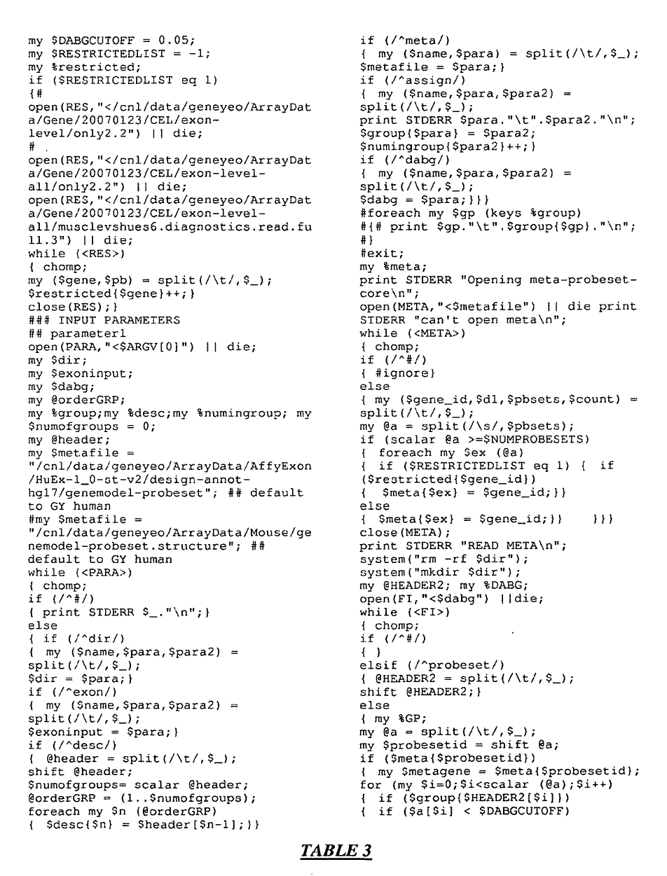

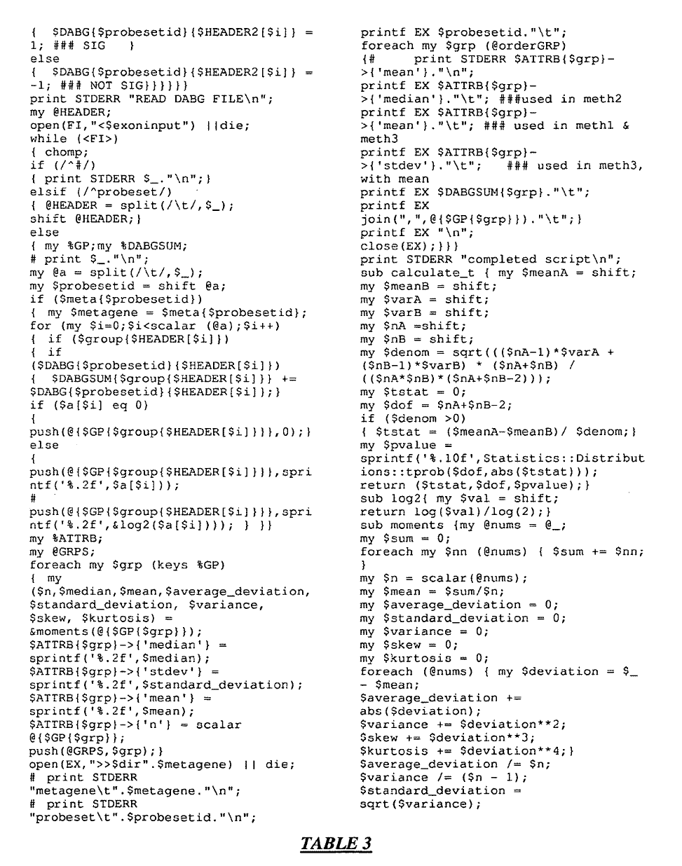

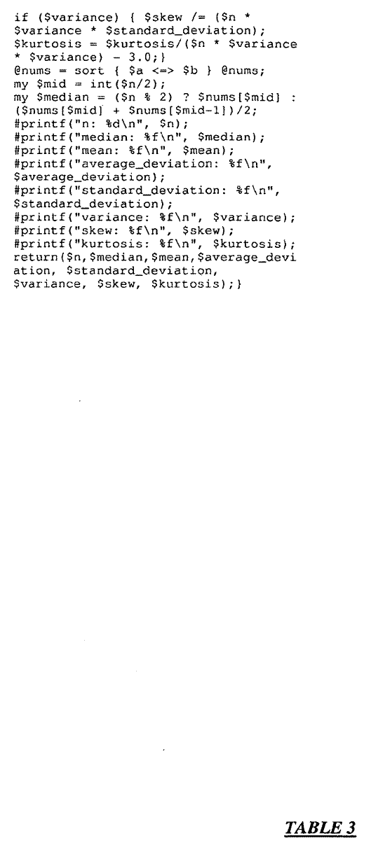

An example of data-processing hardware that can perform analysis according to specific embodiments of the invention is illustrated in FIG. 2. That hardware is operated according to the flowchart of FIG. 1 and/or other methods as described herein. According to this flowchart, a biologic sample is obtained and analyzed on an Affymetrix™ exon array. An output of such an array is a data set, which can be stored on a personal computer such as 700 or a networked server computer such as 720. The output can be processed on 700 and/or 720 to determine data about the biologic samples, and to output that data, e.g., on a display screen 705. In an embodiment, the materials used are undifferentiated embryonic stem cells (Cyt-ES) and multipotent neuroprogenitor cells, for example, central nervous system neurospheres (hCNS-SCns).

### Example General Method

FIG. 1 illustrates a basic flowchart of a method for identifying AS events according to specific embodiments of the invention. At 100, neural progenitors are individually derived from these two lines, processed and hybridized onto the Affymetrix™ exon array 210. Data is obtained at 110. At 120, the data are normalized and signal estimates are obtained using robust multichip analysis. Data are selected for analysis if found to be sufficiently relevant. For example, different characteristics can be used to determine which probe sets to analyze. An embodiment analyzes probe sets only if they were comprised of three or more individual probes, or localized within the exons of the gene models with evidence from at least three different gene models (e.g., mRNA, EST or full length cDNA) and were detected above background in at least one of the cell populations. The background detection can be done using the publicly available Affymetrix™ power tools, or some other similar program.

In the embodiment, alternative spliced exons are detected by finding probe sets that behave unexpectedly in one cell type compared to another, e.g., in the Cyt-ES cells, compared with the nuerospheres benchmark.

Example Experiment Comparing Cyt-ES to hCNS-SCns

Further details of one example experiment are provided below. Cyt-ES was compared to hCNS-SCns to illustrate the invention. Data produced by an Affeymetrix EXON array was first normalized and signal estimates were generated using Robust Multichip Analysis (RMA). The probability that each probeset was detected above background (DABG) was estimated using publicly available Affymetrix Power Tools (APT).

In a particular example experiment, probesets were selected for further analysis if those probesets (i) comprised three or more individual probes; (ii) were localized within the exons of selected gene models with evidence from at least three sources (mRNA, EST, or full-length cDNA); and (iii) were detected above background in at least one of the cell lines. In total, 17,430 gene models in this experiment were represented by probesets that satisfied these criteria. Next it was determined if probeset expression within each gene model was positively correlated for any two cell lines. To do this in this example, we a Pearson correlation coefficient was determined between the vectors of median signal estimates across replicates in Cyt-ES versus hCNS-SCns. The vast majority of genes (0.80%) was found to have probeset-level Pearson correlation coefficients of greater than 0.8 (FIG. 3A).

To confirm the approach, we randomly permuted the association between the median signal estimates and the probesets for each gene in hESCs (or hCNS-SCns) and observed that the distribution of Pearson correlation coefficients for the permuted sets was centered at zero, as expected (FIG. 3A). This indicated that the signal estimates for probesets between hESCs and hCNS-SCns were highly correlated and suggested that a scatter plot of probeset signal estimates between hESCs and hCNS-SCns would reveal a linear relationship for the majority of genes. A robust linear regression was used to determine if some probesets behaved unexpectedly in one cell type compared to the other might in order to identify AS exons.

### 2. Analyzing the Responses from Both Cell

FIG. 3A-F illustrate a REAP method comparing exon array signal estimates from hCNS-SCns and Cyt-ES according to specific embodiments of the invention. FIG. 3(A) illustrates a histogram of Pearson correlation coefficients computed from median signal estimates for probesets between Cyt-ES versus hCNS-SCns for genes (the bars with a peak at the right of the graph). In this example embodiment, genes were required to have more than five probesets localized within the exons in the gene. The bars with a central peak represented Pearson correlation coefficients computed from exons with shuffled signal estimates. FIG. 3(B) illustrates that each probeset contained probeset-level estimates from three replicates (e.g., from three different exon array data sets) labelled, in this case, (a, b, c) in Cyt-ES and (d, e, f) in hCNS-SCns. Use of three replicates for each sample was done for verification and experimental purposes, with a number of further simplifications as described below. In typical embodiments of the present invention, only one replicate of each cell type may be used.

For the three replicate experiments, the five points summarizing the log, probeset-level estimates are indicated by black filled circles in FIG. 3(C). Scatter plots of signal estimates for probesets that were present in at least one cell type (Cyt-ES or hCNS-SCns) for the EHBP1 gene. In this experiment, probesets were considered present if the DABG p-value was <0.05 for all three replicates in the cell type. A regression line derived from robust linear regression according to specific embodiments of the invention with MM estimation (see, e.g., www(.)statsci(.)org/s/mmnl.html) is indicated. Points above the line represent probesets within exons that were enriched in Cyt-ES and points below represent exons that were enriched in hCNS-SCns. Points close to the regression line are not significantly different in Cyt-ES versus hCNS-SCns. Boxed points represented the five-point summary of a probeset that was significantly enriched in Cyt-ES but was skipped in hCNS-SCns. FIG. 3(D) illustrates a histogram of studentized residuals for points from the scatter plot in FIG. 3(C) in EHBP1. FIG. 3(E) illustrates the histogram of studentized residuals for all points for all analyzed probesets (100 bins). FIG. 3(F) illustrates the scatter plot of studentized residuals generated from comparing Cyt-ES versus hCNS-SCns and hCNS-SCns versus Cyt-ES of 5,000 randomly chosen probesets.

In this experiment, a simplification of the multiple replicate data was explored. If we had N replicates in one condition (e.g., of one cell type) and M replicates in the other, we could consider N*M points if we analyzed every possible pairing. For instance, three replicate signal estimates for every probeset per cell line, such as signal estimates a, b, and c in hESCs and d, e, and f in hCNS-SCns, would translate to pairing every signal (d,a), (d,b), (d,c) (f,a), (f,b), (f,c) for linear regression (FIG. 3B). Instead, pairing the signal estimates of all replicates in one condition to the median of the other would only require N+M−1 points. Using robust regression, the regression line for Cyt-ESC versus hCNS-SCns in the EHBP1 gene is illustrated in FIG. 3C. The boxed points belonged to a probeset that was enriched in hESCs but depleted in hCNS-SCns, which was suspected to be due to AS. The difference between the actual and regression-based predicted value, normalized by the estimate of its standard deviation, is called the studentized residuals. Studentized residuals were computed for all probeset pairs in EHBP1, and the histogram depicting their distribution is illustrated in FIG. 3D. As expected, the mean of the distribution was close to zero, and the distribution was approximated by a t-distribution with n−p−1 degrees of freedom, where n was the number of points on the scatter plot, and the number of parameters p was 2. The boxed points had studentized residuals of 1.829, 3.104, 2.634, 3.012, and 2.125 with p-values of 0.034, 0.00119, 0.00477, 0.00158, and 0.01780, respectively, computed based on the t-distribution (FIG. 3C). At a stringent p-value cutoff of 0.01, four of the five studentized residuals were designated as significant “outliers,” indicating that the probeset was “unusual.” RT-PCR confirmed that the exon, represented by the probeset, was indeed differentially included in hESCs and skipped in hCNS-SCns (FIG. 7B). Applying this approach to all gene models revealed that, as expected, the majority of studentized residuals are centered at zero (FIG. 3E). Thus far in the example, our analysis was based on regression of hESCs (y-axis) versus hCNS-SCns (x-axis) (FIG. 3B-3D). However, robust regression as described was not symmetrical, i.e., parameter estimation of y as a function of x was not the same as that of x as a function of y. The negative slope revealed that probesets enriched in hESCs versus hCNS-SCns (positive valued), were expectedly depleted when hCNS-SCns was compared to hESCs (negative valued; FIG. 3F). As our method for predicting candidate alternative exons was based on identification of outliers using robust regression, we named the method REAP.

### 3. Example REAP Method

### Pairwise Simplification

According to specific embodiments of the invention, an optional simplification to the pairing, in which the signal estimates of all replicates in one condition are paired to the median of the other replicate can be performed. 130 shows the simplification pairing; instead of requiring N*M points, this requires only N+M−1 points while still capturing variations in the signal estimates for each probe set. This simplification can become significant for larger numbers of replicates. However, this simplification is optional and will not be present in all embodiments. The simplification avoids pairing of every single signal. When applied to the small point set of FIG. 3A, for example, only the (d,b), (e,a), (e,b), (e,c) and (f,b) are considered after simplification pairing, where b is the median intensity for the Cyt-ES replicate, and d is the median intensity for the hCNS-SCns replicate.

### Scatterplot Data

Based on the simplification pairing, at 140, a scatter plot analysis or data set of all the probe sets for a particular gene or gene model is determined. The scatter plot form that is shown and described with reference to FIGS. 3 and 4 might not actually be created as such, but is explained herein as a visualization tool as will be well understood in the art of statistical analysis. The techniques described herein can determine the outliers without actually determining the plot. A exemplary plot is shown in FIG. 3B, using the format of FIG. 3A, with the hCNS-SCns on the x axis and Cyt-ES on the y axis. Each point on the scatter plot represents the extent of inclusion of an exon in the embryonic stem cells and in the hCNS-SCns. In one example, FIG. 3C can represent a scatter plot of all probesets of the EHBP1 (E H domain binding protein, RefSeq identifier NM_{—}015252) in the format described. Each probeset was represented by 5 points of log-transformed (base 2) values; and each point on the scatter plot reflected the extent of inclusion of an exon in hESCs and in hCNS-SCns (FIG. 3C).

### Robust Regression

The scatter-plot data and further regression analysis can be further understood as follows. A response variable yᵢⱼ is defined which represents the log₂ expression of probeset i in cell type j to explanatory variables xᵢₖ which is the log₂ expression of probeset I in cell type k. For example, j could be Cyt-ES and k could be hCNS-SCns, as illustrated in FIG. 3. While classic linear regression by least squares estimation could be used to determine a linear regression, such procedure may be biased because the least squares prediction may be strongly influenced by the outliers and this may lead to masking the outliers.

At 150, instead of using a least squares based linear regression model, an M-estimation robust regression technique is used to estimate the line 300 in FIG. 3B. Robust regression is a form of regression analysis that is more statistically oriented than classical regression analysis. A number of techniques are know for performing robust linear regression and can be applied to a dataset such as that illustrated in FIG. 3. The source code included herein comprises instructions and scripts for well-known statistical logic packages that can perform a robust linear regression according to specific embodiments of the invention.

Mathematically, M estimation may be carried out as a minimization of

$${\sum }_{i = 1}^{n} ρ ( {x}_{i} , θ ) ,$$

where ρ is a function.
The solutions

$$\hat{θ} = {argmin}_{θ} ( {\sum }_{i = 1}^{n} ρ ( {x}_{i} , θ ) )$$

are called M-estimators (“M” for “maximum likelihood-type”)
The function ρ, or its derivative, ρ, can be chosen in such a way to bias toward data from the assumed distribution, and away from data/model that is, in some sense, close to the assumed distribution.
This minimization of the equation can be done iteratively in this embodiment. Another alternative is to differentiate with respect to θ and solve for the root of the derivative. The iteration can use standard function optimization algorithms, such as Newton-Raphson. An embodiment uses iteratively re-weighted least squares algorithm. The iteration starts from a robust starting point, such as the median.

While the present embodiment describes using an M-estimator, other types of robust estimators could be used, including L-estimators, R-estimators and S-estimators. In general, any regression technique that does not hide the outliers can be used for this purpose.

### Fitting

Fitting is performed using an iterated related least squares analysis. The assumption made is that most of the points are correct, that is most of the exons are constitutively spliced. Thus, robust regression finds the line that is least dependent on the outliers.

### Finding Outliers

The outliers are found at 160, and are assumed to be the alternatively spliced exons.

The outliers are checked at 170. The techniques described herein use a t-distribution which analyzes the samples based on an estimate of standard deviation. A studentized residual forms the difference between the actual value and the value correctly predicted by the regression line 300, normalized by an estimate of the standard deviation. The studentized residuals are computed for all the probe set pairs. FIG. 3C depicts the distribution of these studentized residuals. Since this is in effect a random function, the mean of the distribution is close to zero, and it can be approximated by a t-distribution with an n−p−1° of freedom, where n is the number of points on the scatter plot, and the number of parameters p=2.

The boxed points 305 in FIG. 3B have studentized residuals respectively of 1.829, 3.104, 2.634, 3.012, and 2.125, with “p-values” of 0.00119, 0.00477, 0.00158 and 0.01780, respectively, based on a t-distribution. A p value represents the probability that the signal intensity is part of the null distribution. The p-value measures the statistical significance of any point to the distribution. For example, the p-value represents the probability that, given that the null hypothesis is true, T will assume a value as or more unfavorable to the null hypothesis as the observed value. The assumptions made were substantiated by the inventors through experiment by observing results. A stringent p-value cut off can be used herein of 0.01, based on review of actual data sets. This allows designating four of the five studentized residuals as being significant outliers, indicating that the probe set is likely to be unusual.

### Removing False Positives

Step 180 generically represents removing false positives, as part of the finding outliers. Experimental validations of the predictions have identified three main sources of false positives from the robust regression. Probeset signal estimates that are poorly correlated do not work well with this technique. The correlation can be evaluated using Pearson correlation coefficients.

The Pearson coefficient forms a measure of the correlation of two variables x and y on the same object or organism. This correlation can be mathematically defined as the sum of the products of the standard scores of the two measures divided by the degrees of freedom:

$$r = \frac{\sum {z}_{x} {z}_{y}}{n - 1}$$

Note that this formula assumes the Z scores are calculated using standard deviations which are calculated using n−1 in the denominator.

The result obtained is equivalent to dividing the covariance between the two variables by the product of their standard deviations.

Based on experimental review, it was found that more than 80% of the genes had probe set level Pearson correlation coefficients of greater than 0.8. It was also found that the distribution of these Pearson correlation components was centered at zero or close to zero. From this, it was generalized that a scatter plot of the estimates would reveal a linear relationship for the majority of genes.

### Pearson Correlation Coefficient Cut Off.

A first false positive is avoided by selecting a Pearson correlation coefficient cut off. Empirically, an embodiment determines 0.6 as being a Pearson correlation coefficient, below which, the gene is not amenable to the REAP protocol. The gene sample to be removed at 180 if its Pearson correlation coefficient is less than 0.6.

### High Leverage Points and High Influence Points

High leverage points and high influence points also have tended to form false positives. These points are determined by metrics.

According to an embodiment, the metrics are obtained by determining the influence, and the leverage, of the point. FIG. 4A shows classifying points as outliers if they have a large studentized residual (P<0.01) and low leverage, see boxed point a. The boxed point b is a high leverage point that has a large studentized residual and a high leverage. The boxed point c is a high influence point that has a high studentized residual, high leverage, and high influence.

FIG. 4B shows boxed points that are high leverage, while FIG. 4C shows the boxed points that are high influence.

Four of the five points in FIG. 4B were experimentally verified to be false positives. Therefore, while not all of these high leverage points will be false positives, generally points which are significant outliers and do not meet these criteria are selected to be putative alternative splicing events.

For an embodiment, leverage assesses how far away a value of the independent variable is from its mean value. When the value is further from the mean value, it has more leverage. A point in this embodiment can be considered to have high leverage, when the leverage hᵢ (of the ith point)>3p/n, where p is the number of variables and n is the number of points.

The leverage of the ith point can be expressed as:

hᵢ=n⁻¹+(xᵢ−μₓ)²/(sₓ²(n−1)), where μₓ=Σxᵢ²/n.

The influence of the points is related to covariance. A covariance ratio is formed as a ratio of the determine of the covariance matrix with the entire sample. A covariance that is larger than 1 implies the point is closer than typical to the regression line. Accordingly, a point is considered to have high influence if |covᵢ−1|>3p/n

### Exon Array Analysis

Preparation of biologic samples and initial data capture and analysis of the Exon expression data may be done according to any number of procedures known in the art as well as those described herein and in the included references. In one example, the Affymetrix™ Power Tools (APT) suite of programs was obtained from the worldwide web at affymetrix.com/support/developer/powertools/index.affx. Exon (probeset) and gene-level signal estimates were derived from the CEL files by RMA-sketch normalization as a method in the apt-probeset-summarize program. To determine if the signal intensity for a given probeset is above the expected level of background noise, we utilized the DABG (detection above background) quantification method available in the apt-probeset-summarize program as part of the Affymetrix™ Power Tools (APT). Briefly, DABG compared the signal for each probe to a background distribution of signals from anti-genomic probes with the same GC content. The DABG algorithm generated a p-value representing the probability that the signal intensity of a given probe is part of the background distribution. A probeset with a DABG p-value lower than 0.05 was considered to be detected above background. The statistic t_{hCNS-SCnsESC}=(μ_{hCNS-SCns}−μ_{ESC})/sqrt(((n_{hCNS-SCns}−1)σ²_{hCNS-SCns}+(n_{ESC}−1)σ²_{ESC})(n_{FNSC}+n_{ESC}))/((n_{FNSC}+n_{ESC})(n_{hCNS-SCns}+n_{ESC}−2))), where n_{hCNS-SCns} and n_{ESC} were the number of replicates, μ_{hCNS-SCns} and μ_{ESC} were the mean, and σ²_{hCNS-SCns} and σ²_{ESC} were the variances of the expression values for the two datasets was used to represent the differential enrichment of a gene using gene-level estimates in hCNS-SCns relative to hESCs. Multiple hypothesis testing was corrected by controlling for the false discovery rate (e.g., via Benjamini-Hochberg).

### 4. Specific Example Implementation Using REAP to Identify AS Events

In order to provide further understanding of the invention, a particular example method is described below. It will be understood that this example is illustrative of the general methods of the invention and that many variations in parameters and steps in the analysis will be understood by those of skill in the art.

In a particular example embodiment, the log₂ signal estimate xᵢⱼ for probeset i in cell-type j was checked to satisfy the following two conditions, otherwise the probeset was discarded: (i) 2<xᵢⱼ<10,000 for all conditions/cell-types j; and (ii) DABG p-value<0.01 for all replicates in at least one condition/cell-type j. A gene or gene-model had to have five probesets that satisfied the two conditions above in order to be considered for robust regression analysis in this example.

After determining the data points for a gene model to be analyzed, the robust regression method rlm in R-package “MASS” (version 6.1-2, see e.g., 11. W. N. Venables and B. D. Ripley. Modern Applied Statistics with S-PLUS. Springer, N.Y., second edition, 1997.) with M-estimation and a maximum iteration setting of 30 was used to estimate the linear function yᵢ=αxᵢ+β. For each probeset, the method computed the error term eᵢ which was the difference between the actual value yᵢ and the estimated value ξᵢ, from the estimated function ξᵢ=Axᵢ+B, where A and B were estimates of α and β. The error term variance was estimated by sₑ²=Σeᵢ²/(n−p), which was used to estimate the variance of the predicted value, s_{ξi}²=sₑ²(n⁻¹+xᵢ−μₓ)²/sₓ²(n−1)). Here, n referred to the number of points (generated for each gene), and p referred to the number of independent variables (p=2 in our method); and μₓ=Σxᵢ²/n; sₓ²=n⁻¹Σ(xᵢ−μₓ)².

Following Belsley et al. (Belsley et al., Regression Diagnostics: Identifying Influential Data and Sources of Collinearity 1980 John Wiley and Sons, New York), leverage hᵢ of the i^{th} point was determined by hᵢ=n⁻¹+(xᵢ−μₓ)²(n−1). A point was considered to have high leverage if hᵢ>3p/n.

The covariance ratio, covᵢ=(sᵢ²/sᵣ²)P/(1-hᵢ), is the ratio of the determinant of the covariance matrix after deleting the i^{th} observation to the determinant of the covariance matrix with the entire sample. A point was considered to have high influence if |covᵢ−1|>3p/n.

The studentized residuals, rstudentᵢ=eᵢ/(s₍ᵢ₎²(1−hᵢ)^{0.5}), where s₍ᵢ₎²=(n−p)sₑ²/(n−p−1)−eᵢ²/(n−p−1)(1−hᵢ), the error term variance after deleting the i^{th} point. As rstudentᵢ was distributed as Student's t-distribution with n−p−1 degrees of freedom, each rstudentᵢ point was associated with a p-value. A point was identified as an ‘outlier’ if p<0.01.

### Identification of Motifs

The enrichment score of a sequence element of length k (k-mer) in one set of sequences (set 1) versus another set of sequences (set 2) was represented by the non-parametric χ² statistic with Yates correction, computed from the two by two contingency table, T(T₁₁: number of occurrences of the element in set 1; T₁₂: number of occurrences of all other elements of similar length in set 1; T₂₁: number of occurrences of element in set 2; T₂₂ number of occurrences of all other elements of similar length in set 2. All elements had to be greater than 5. To correct for multiple hypothesis testing, p-values were multiplied by the total number of comparisons.

### Reap[+j]

Experimental validation of REAP[+] exons suggested a high specificity at the expense of relatively moderate sensitivity. High false-positive rates may arise from cross-hybridization effects that remained unaccounted for, which is likely a design issue for the arrays. However, our specificity of 77% at the cutoff of two significant outliers per probeset allows us to estimate that at least 1,336 of 1,737 REAP[+] exons are true alternative splicing events that distinguish NPs and hESCs. On average, 7% of all human exons have been estimated by transcript data to undergo alternative splicing; thus REAP's validation rate of 60% at the cutoff of two is 73-fold (60/7) higher than expected.

The methods of the present invention where further used to determine nine novel alternative splicing events that distinguish hESCs and NPs. In addition, it was observed that the alternative splicing patterns in hCNS-SCns were not always similar to those of the derived NPs. Thus, it is demonstrated that alternative splicing is able to distinguish derived NPs and hCNS-SCns. A strong exception was the alternative exon in the SLK gene, encoding a serine/threonine kinase protein, which was strongly included in hESCs i.e. the exon-excluded isoform was not present in hESCs compared to NPs, as well as in a variety of differentiated tissues. Closer inspection of the REAP[+] validated alternative splicing exon in the SLK gene revealed strong conservation in the intronic region flanking the exon, a hallmark feature of alternative splicing exons (Sugnet, 2004 Pac Symp Biocomput: 66-77; Yeo, 2005 #Proc Natl Acad Sci USA 102 (8):2850; Sorek, 2003 Genome Re 13(7): 1631). A published study analyzing the expression patterns of the SLK gene suggested a potential functional role during embryonic development and in the adult central nervous system (Zhang, 2002 Brain Res Dev Brain REs 139(2): 205); however, to our knowledge, our identification of the SLK alternative exon is the first report of a hESC-specific alternative splicing pattern. Moreover, Gene Ontology (G O) analysis suggested that genes containing REAP[+] exons were enriched in serine/threonine kinase activity, of which SLK is a family member.

It was experimentally found that REAP[+] exons were underrepresented in genes that were transcriptionally different in expression in hESCs and NPs.

The studies identified potential cis-regulatory intronic elements conserved and enriched proximal to the REAP[+] exons. In particular, the FOX1 binding site, GCUAG, was conserved and enriched in the flanking introns of a subset of REAP[+] exons. REAP and the analysis of alternative splicing has revealed new and unanticipated insights into human embryonic stem cell biology and their transition to neural progenitor cells.

### 5. Example System

Maintenance and Differentiation of hESCs and hCNS-SCns

hESC line Cy203 (Cythera Inc.) was cultured as previously described ((Muotri et al, 2005 Proc Natl Acad Sci. USA 102 (51): 18644-18648). To differentiate into neuroepithelial precursor cells, colonies were manually isolated from mouse embryonic fibroblasts (MEFs) and cut in small pieces. These pieces were transferred to a T75 flask with hESCs differentiation media (same hESC medium but 10% KSR and no FGF-2). Medium was changed the next day by transferring the floating hESC aggregates to a new flask. After culturing for a week, the hESC cell aggregates formed mature embroid bodies (EBs; ˜10 um round clusters with dark centers). EBs were plated on a coated 10-cm dish in hESC differentiation media. The next day, the medium was changed to DMEM/F12 supplemented with ITS and fibronectin. Medium was changed every other day for a week or until the cells formed rosette-like columnar structures that were isolated manually. These structures were then transferred to coated dishes in neural induction medium (DMEM/F12 supplemented with N2 and FGF-2) for a week. Elongated single cells were separated from leftover aggregates using non-enzymatic dissociation. After one to two passages, the cells formed a monolayer of homogeneous NPs (negative for Sox 1 immunostaining). Upon confluence, cells will form neurospheres that can also be isolated from the neuroepithelial precursor cells (positive for Sox1 immunostaining). At any of these two stages, pan-neuronal differentiation can be achieved after three to four weeks. hESC line HUES6 was cultured on MEF feeders as previously described (see the worldwide web at mcb.harvard.edu/melton/hues/) or on GFR matrigel coated plates. Cells grown on matrigel were grown in MEF-conditioned medium and FGF-2 was used at 20 ng/mL instead of 10 ng/mL for cells grown on MEFs. To differentiate neuroepithelial precursors, colonies were removed by treatment with collagenase I V (Sigma) and washed three times in growth media. The pieces of colonies were resuspended in HUES growth media without FGF2 in an uncoated bacterial Petri dish to form EBs. After one week, EBs were plated on polyornathine/laminin coated plates in DMEM/F12 supplemented with N2 and FGF2. Rosette structures were manually collected and enzymatically dissociated with TryPLE (Invitrogen), plated on polyornathine/laminin coated plates and grown in DMEM/F12 supplemented with N2 and B27-RA and 20 ng/mL FGF-2. Cells could be grown as a monolayer for up to at least ten passages. Cells were Sox1- and nestin-positive and readily differentiated into neurons upon withdrawal of FGF-2. Human central nervous system stem cell line FBR1664 (StemCells Inc) which is referred to as hCNS-SCns in the main text was cultured as previously described (Uchida, 2000 Proc Natl Acad Sci USA 97(26):14720-14725). The cells were cultured in medium consisting of Ex Vivo 15 (BioWhittaker) medium with N2 supplement (GIBCO), FGF2 (20 ng/mL), epidermal growth factor (20 ng/mL), lymphocyte inhibitory factor (10 ng/mL), 0.2 mg/ml heparin, and 60 ug/mL N-acetylcysteine. Cultures were fed weekly and passaged at about two to three weeks using collagenases (Roche). The following antibodies and corresponding dilutions were utilized for the immunohistochemical analysis of marker genes in Cyt-ES and HUES6-ES: Sox2 (chemicon, 1:500), October 4 (Santa Cruz, 1:500), Sox1 (Chemicon, 1:500), Nestin (Pharmingen, 1:250); hCNS-SCns: Sox1 (1:500), Sox2 (Chemicon, 1:200), Nestin (Chemicon, 1:200).

### RNA Preparation and Array Hybridization

Total RNA was extracted, and labeled cDNA targets were generated from three independent preparations of each of the five cell types, namely Cyt-ES, HUES6-ES, Cyt-NP, HUES6-NP, and hCNS-SCns. To facilitate downstream analyses, instead of utilizing the metagene sets available from the manufacturers, we generated our own gene models by clustering alignments of ESTs and mRNAs to annotated known genes from the University of California Santa Cruz (UCSC) Genome Browser Database. After hybridization, scanning, and extraction of signal estimates for each probeset on the exon arrays, gene-level estimates were computed based on our gene models using available normalization and signal estimation software from Affymetrix. For every gene, a t-statistic and corresponding p-value were computed representing the relative enrichment of the expression of the gene in hESC versus NP, such as in Cyt-ES versus Cyt-NP. After correcting for multiple hypothesis testing using the Benjamini-Hochberg method, a p-value cutoff of 0.01 was used to identify enriched genes. Close inspection of all pairs of hESC-NP comparisons revealed a generally significant overlap from 31% to 85% of the smaller of two compared sets of enriched genes (see FIG. S1). Thus for the purpose of identifying overall pluripotent and neural lineage-specific genes, the collective set of NPs (Cyt-NP, HUES6-NP, and hCNS-SCns) was compared to the collective set of hESCs (Cyt-ES and HUES6-ES). To summarize, firstly immunohistochemical and RT-PCR reflected expected molecular and biological differences evidence validated that the cells exhibited expected charac-between hESCs and NPs, we sought to identify AS events.

Total RNA from cells was processed as follows. Cells were lysed in 1 mL of RNA-bee (Teltest, Friendswood, Tex., U.S.A.). The RNA was isolated by chloroform extraction of the aqueous phase, followed by isopropanol precipitation as per the manufacturer's instructions. The precipitated RNA was washed in 75% ethanol and eluted with DEPC-treated water. Five ug of RNA was treated with R Q1 DNAase (Promega) according to the manufacturer's instructions. One ug of total RNA for each sample was processed using the Affymetrix™ GeneChip Whole Transcript Sense Target Labeling Assay (Affymetrix, Inc., Santa Clara, Calif.). Ribosomal RNA was reduced with the RiboMinus Kit (Invitrogen). Target material was prepared using commercially available Affymetrix™ GeneChip WT cDNASynthesis Kit, WT cDNA Amplification Kit, and WT Terminal Labeling Kit (Affymetrix, Inc., Santa Clara, Calif.) as per manufacturer's instructions. Hybridization cocktails containing about 5 ug of fragmented and labeled DNA target were prepared and applied to GeneChip Human Exon 1.0 ST arrays. Hybridization was performed for 16 hours using the Fluidics 450 station. Arrays were scanned using the Affymetrix™ 3000 7G scanner and GeneChip Operating Software v1.4 to produce .CEL intensity files.

### Detection of Alternative Splicing by RT-PCR

cDNAs were generated from total RNA with Superscript III reverse transcriptase (Invitrogen Inc.). PCR reactions were performed with primer pairs designed for alternative splicing targets (annealing at 58° C. and amplification for 30 or 35 cycles). PCR products were resolved on either 1.5% or 3% agarose gel in TBE. The Ethidium Bromide-stained gels were scanned with Typhoon 8600 scanner (Molecular Dynamics Inc.) for quantitation. The number of true positives (TP; false negatives, FN) was computed as the number of REAP[+] (REAP[−]) exons that were validated by RT-PCR as alternative splicing. The number of true negatives, TN (or false positives, FP) was computed as the number of REAP[−] (REAP[+]) exons that were validated by RT-PCR as constitutively spliced. The true (false) positive rate was computed as TP (FP) divided by the total number of REAP[+] exons in the experimentally validated set. The true (false) negative rate was computed as the TN (FN) divided by the total number of REAP[−] exons in the experimentally validated set. Sensitivity was computed as TP/(TP+FN) and specificity was computed as TN/(FP+TN).

### Sequence Databases

Genome sequences of human (hg17), dog (canFam1), rat (rn3) and mouse (mm5) were obtained from the University of California Santa Cruz (UCSC), as were the whole-genome MULTIZ alignments (Karolchik, 2003 Nucleic Acids REs 31(1):51-54). The lists of known human genes (known Gene containing 43,401 entries) and known isoforms (known Isoforms containing 43,286 entries in 21,397 unique isoform clusters) with annotated exon alignments to human hg17 genomic sequence were processed as follows. Known genes that were mapped to different isoform clusters were discarded. All mRNAs aligned to hg17 that were greater than 300 bases long were clustered together with the known isoforms. Genes containing less than three exons were removed from further consideration. A total of 2.7 million spliced expressed sequence tags (ESTs) were mapped onto the 17,478 high-quality genes to infer alternative splicing. Exons with canonical splice signals (GT-AG, AT-AC, GC-AG) were retained, resulting in a total of 213,736 exons. Of these, 197,262 (92% of all exons) were constitutive exons, 13,934 exons (7%) had evidence of exon-skipping, 1615 (1%) exons were mutually-exclusive alternative events, 5,930 (3%) exons had alternative 3′ splice sites, 5,181 (2%) exons had alternative 5′ splice sites, and 175 (<1%) exons overlapped another exon, but did not fall into the above classifications. A total of 324,139 probesets from the Affymetrix™ Human Exon 1.0 ST array were mapped to 208,422 human exons, representing 17,431 genes. These probesets were used to derive gene and exon-level signal estimates from the CEL files. The four-way mammalian (four-mammal) whole-genome alignment (hg17, canFam1, mm5, rn3) was extracted from the eight-way vertebrate MULTIZ alignments (hg17, panTrol1, mm5, rn3, canFam1, galGal2, fr1, danRer1) obtained from the UCSC genome browser. Four-way mammal alignments were extracted for all internal exons, and 400 bases of flanking intronic sequence, resulting in a total of 161,731 conserved internal exons. A total of 145,613 (90% of total) conserved internal exons were constitutive exons, 13,653 exons (8%) had evidence of exon-skipping, 1576 exons were mutually exclusive alternative events, 5,818 exons had alternative 3′ splice sites, 5,046 exons had alternative 5′ splice sites, and 168 exons overlapped another exon.

The general structure and techniques, and more specific embodiments which can be used to effect different ways of carrying out the more general goals are described herein. Although only a few embodiments have been disclosed in detail above, other embodiments are possible and the inventor (s) intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, while Affymetrix™ exon arrays are described in the embodiments, other embodiments may use other kinds of readout. For example, a high-throughput sequencing technique like Solexa can be used to identify sequence tags that are later mapped to exons. The techniques can be applied directly to the Solexa sequenced tags; using the REAP after converting digital counts to a sort of score for each exon. Then the scores can be plotted on a scatter plot and the techniques described herein are used for analysis. Moreover, as described herein, the scatter plot is a visualization tool, and the computer techniques described herein need not actually make any kind of scatter plot.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The computer may be an Intel (e.g., Pentium or Core 2 duo) or AMD based computer, running Windows XP or Linux, or may be a Macintosh computer. The computer may also be a handheld computer, such as a PDA, cellphone, or laptop.

The programs may be written in C or Python, or Java, Brew or any other programming language. The programs may be resident on a storage medium, e.g., magnetic or optical, e.g. the computer hard drive, a removable disk or media such as a memory stick or S D media, wired or wireless network based or Bluetooth based Network Attached Storage (NAS), or other removable medium, or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

### 6. EXON Detection

Exons of the invention can be detected by any available nucleic acid detection method, including Southern or northern hybridization, hybridization to a probe or array, amplification, or the like. For example, in one embodiment, an alternate splicing isoform is detected by hybridization of a probe comprising an exon sequence, or exon sequences, e.g., those noted herein of interest to a nucleic acid (e.g., mRNA or cDNA). For example, the nucleic acid can be from a cell type of interest, e.g., an embryonic stem cell, a neuroprogenitor cell, or the like. Typical hybridization formats can include Southern analysis, northern analysis, or the like. Probes can correspond to the exon sequences noted herein (e.g., probes can include sequences that are at least partially complimentary to a given exon or splice site). Details regarding hybridization formats can be found in Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc.

Array based hybridization provides one convenient hybridization format to detect splicing isoforms of interest, e.g., using probes corresponding to the exons noted herein. Array formats and technology is reviewed in, e.g., Kimmel and Oliver (eds) (2006) DNA Microarrays Part A: Array Platforms & Wet-Bench Protocols, Volume 410 (Methods in Enzymology) Academic Press; 1st edition ISBN-10: 0121828158; Kimmel and Oliver (2006) DNA Microarrays, Part B: Databases and Statistics, Volume 411 (Methods in Enzymology) Academic Press; 1st edition ISBN-10: 0121828166; Primrose and Twyman (2006) Principles of Gene Manipulation and Genomics Wiley-Blackwell, 7th edition 1SBN-10: 1405135441; Gibson and Muse (2004) A Primer of Genome Science, 2nd Edition Sinauer Associates; 2nd edition ISBN-10: 0878932321; Lausted et al. (2004) POSaM: a fast, flexible, open-source, inkjet oligonucleotide synthesizer and microarrayer Genome Biol. 5(8): R58.Published online 2004 Jul. 27. doi: 10.1186/gb-2004-5-8-r58; Draghici (2003) Data Analysis Tools for DNA Microarrays Chapman & Hall/CRC; ISBN-10: 1584883154; Stekel (2003) Microarray Bioinformatics Cambridge University Press; 1st edition # ISBN-10: 052152587X; Baldi et al. (2002) DNA Microarrays and Gene Expression: From Experiments to Data Analysis and Modeling Cambridge University Press; 1st edition ISBN-10: 0521800226; and DNA Microarrays: Gene Expression Applications (2001) B. R. Jordan (Editor) Springer; 1st edition ISBN-10: 3540415076.

In one class of embodiments, detection includes amplifying the exon, or a sequence associated therewith (e.g., an mRNA, cDNA, an exon flanking sequence, or the like) and detecting the resulting amplicon. For example, amplifying can include a) admixing an amplification primer or amplification primer pair with a nucleic acid alternative splicing isoform, isolated from the organism or biological sample. The primer or primer pair can be complementary or partially complementary to a region proximal to or including a splice junction, capable of initiating nucleic acid polymerization by a polymerase on the nucleic acid template. The primer or primer pair is extended in a DNA polymerization reaction comprising a polymerase and the template nucleic acid to generate the amplicon. In certain aspects, the amplicon is optionally detected by a process that includes hybridizing the amplicon to an array, digesting the amplicon with a restriction enzyme, or real-time PCR analysis. Optionally, the amplicon can be fully or partially sequenced, e.g., by hybridization. Typically, amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. Other technologies can be substituted for amplification, e.g., use of branched DNA (bDNA) probes. Techniques for amplification can be found in Sambrook et al, Ausubel et al and, e.g., in PCR Protocols A Guide to Methods and Applications (Innis et al. eds) Academic Press Inc. San Diego, Calif. (1990) (Innis), Chen et al. (ed) PCR Cloning Protocols, Second Edition (Methods in Molecular Biology, volume 192) Humana Press; and in Viljoen et al. (2005) Molecular Diagnostic PCR Handbook Springer, ISBN 1402034032.

Any isoform can also be sequenced, using standard techniques such as those noted in Sambrook or Ausubel, by using high-throughput DNA sequencing systems (reviewed in, e.g., Chan, et al. (2005) “Advances in Sequencing Technology” (Review) Mutation Research 573: 13-40). See, also, e.g., Hodges, et al. (2007) “Genome-wide in situ exon capture for selective resequencing.” Nat Genet 39: 1522-1527; Olson M (2007) “Enrichment of super-sized resequencing targets from the human genome.” Nat Methods 4: 891-892; and Porreca, et al. (2007) “Multiplex amplification of large sets of human exons.” Nat Methods 4: 931-936.

In general, a wide variety of nucleic acids can be analyzed for the presence of particular exons in the methods and compositions herein. These include RNA, cDNA, cloned nucleic acids (DNA or RNA), expressed nucleic acids, genomic nucleic acids, amplified nucleic acids, and the like. Details regarding nucleic acids, including detection of nucleic acids, isolation, cloning and amplification can be found, e.g., in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc; Kaufman et al. (2003) Handbook of Molecular and Cellular Methods in Biology and Medicine Second Edition Ceske (ed) CRC Press (Kaufman); and The Nucleic Acid Protocols Handbook Ralph Rapley (ed) (2000) Cold Spring Harbor, Humana Press Inc (Rapley).

Cell culture media appropriate for growing cells that comprise splicing isoforms are set forth in the previous references and, additionally, in Atlas and Parks (eds) The Handbook of Microbiological. Media (1993) CRC Press, Boca Raton. F L. Additional information for cell culture is found in available commercial literature such as the Life Science Research Cell Culture Catalogue (1998) from Sigma-Aldrich, Inc (St Louis, Mo.) (“Sigma-LSRCCC”) and, e.g., the Plant Culture Catalogue and supplement (e.g., 1997 or later) also from Sigma-Aldrich, Inc (St Louis, Mo.) (“Sigma-PCCS”). The culture of animal cells is described. e.g., by Freshney (2000) Culture of Animal Cells: A Manual Of Basic Techniques John Wiley and Sons, N Y.

In addition to other references noted herein, a variety of purification/protein purification methods are well known in the art and can be applied to analysis and purification of proteins corresponding to splicing isoforms, isolation of antibodies that are isoform specific, and the like. Relevant protein purification and antibody isolation methods are taught in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, N Y; Walker (1996) The Protein Protocols Handbook Humana Press, N J; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, N Y; Janson and Ryden (1998) Protein Purification: Principles, High Resolution Methods and Applications, Second Edition Wiley-VCH, N Y; and Walker (1998) Protein Protocols on CD-ROM Humana Press, N J; and the references cited therein.

### 7. Embodiment in a Programmed Information Appliance

FIG. 2 As will be understood to practitioners in the art from the teachings provided herein, the invention can be implemented in hardware and/or software. In some embodiments of the invention, different aspects of the invention can be implemented in either client-side logic or server-side logic. As will be understood in the art, the invention or components thereof may be embodied in a fixed media program component containing logic instructions and/or data that when loaded into an appropriately configured computing device cause that device to perform according to the invention. As will be understood in the art, a fixed media containing logic instructions may be delivered to a user on a fixed media for physically loading into a user's computer or a fixed media containing logic instructions may reside on a remote server that a user accesses through a communication medium in order to download a program component.

FIG. 2 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection.

The invention also may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

### 8. Other Embodiments

The invention has now been described with reference to specific embodiments. Other embodiments will be apparent to those of skill in the art. In particular, a user digital information appliance has generally been illustrated as a personal computer. However, the digital computing device is meant to be any information appliance for interacting with a remote data application, and could include such devices as a digitally enabled television, cell phone, personal digital assistant, laboratory or manufacturing equipment, etc. It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this application, including any references filed as part of an Information Disclosure Statement, are incorporated by reference in their entirety.

The general structure and techniques, and more specific embodiments which can be used to effect different ways of carrying out the more general goals are described herein.

Although only a few embodiments have been disclosed in detail above, other embodiments are possible and the inventor (s) intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, While Affymetrix™ exon arrays are described in the embodiments, other embodiments may use other kinds of readout. For example, a high-throughput sequencing technique like Solexa can be used to identify sequence tags that are later mapped to exons. The techniques can be applied directly to the Solexa sequenced tags; using the REAP after converting digital counts to a sort of score for each exon. Then the scores can be plotted on a scatter plot and the techniques described herein are used for analysis. Moreover, as described herein, the scatter plot is a visualization tool, and the computer techniques described herein need not actually make any kind of scatter plot.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The computer may be an Intel (e.g., Pentium or Core 2 duo) or AMD based computer, running Windows XP or Linux, or may be a Macintosh computer. The computer may also be a handheld computer, such as a PDA, cellphone, or laptop.

The programs may be written in C or Python, or Java, Brew or any other programming language. The programs may be resident on a storage medium, e.g., magnetic or optical, e.g. the computer hard drive, a removable disk or media such as a memory stick or S D media, wired or wireless network based or Bluetooth based Network Attached Storage (NAS), or other removable medium, or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

What is claimed:

1. A method of detecting alternative splice (AS) exons between two biologic samples, the method comprising:
receiving exon expression data from two different sample materials of at least one exon set of interest;
said exon expression data comprising expression values of at least three different exons;
performing robust regression analysis of said exon expression data;
wherein said robust regression analysis determines a linearized regression while reducing an impact of any outliers to said linearized regression;
detecting said outliers;
analyzing said outliers to detect false-positive outliers; and
outputting indications of said outliers that are not false positive outliers, said indications identifying one or more exons that are alternatively spliced between said two samples.

2. The method according to claim 1 wherein said samples are from different cellular developmental stages.

3. The method according to claim 1 wherein said samples include undifferentiated cells and differentiated cells.

4. The method according to claim 1 wherein said exon expression data is data captured from exon expression arrays.

5. The method according to claim 1 wherein said exon expression data is data read from one more sequence libraries.

6. The method according to claim 1 wherein said exon expression data is data determined by sequencing and/or hybridization of DNA and/or RNA.

7. The method according to claim 1 further comprising:
normalizing said exon expression data prior said robust regression.

8. The method according to claim 1 further comprising:
using multiply replicates of data sets for each sample;
simplifying a pairing between exon expression data from two sets of multiple replicates separate materials, to avoid requiring pairing between each value from each of the two separate sample materials by pairing between exon expression data of one sample material, and a median of exon expression data from the other sample material.

9. The method according to claim 1 wherein said analyzing said outliers to detect false-positive outliers comprises one or more selected from the group consisting of:
removing values whose Pearson correlation coefficient is less than a predetermined amount;
removing values that have a studentized residual greater than a specified amount; and
removing values that have a leverage that is greater than a specified amount.

10. The method according to claim 1 further wherein:
said two biologic samples comprise pluripotent human embryonic stem cells (hESCs) and multipotent neural progenitor cells (NPs); and
said outliers that are not false positive outliers identify exon expressions that are able to predictively distinguish between pluripotent human embryonic stem cells (hESCs) and multipotent neural progenitor cells (NPs).

11. A method of detecting post RNA-transcription events (e.g., alternative splicing (AS) events or RNA degradation) that are different between first and second biologic samples, the method comprising:
receiving extracted exon signal estimates from at least two biologic samples indicating exon presence and/or expression for a very large exon data set;
determining one or more gene models;
computing gene-level estimates from said extracted exon signal estimates for said gene models;
for a gene, determining a t-statistic and a corresponding p-value representing relative enrichment of expression of said gene between said first sample versus said second sample;
applying a p-value cutoff to identify enriched genes;
for enriched genes, selecting probesets that (i) comprised three or more individual probes; (ii) were localized within the exons of said gene models; and (iii) were detected above background in at least one of the samples lines;
performing a robust regression analysis of said selected probesets to determine if some probesets behaved unexpectedly between said first sample and said second sample to identify AS exons;
wherein said robust regression analysis determines a linearized regression while reducing an impact of any outliers to said linearized regression;
detecting said outliers;
analyzing said outliers to detect false-positive outliers; and
outputting indications of said outliers that are not false positive outliers, said indications identifying one or more probesets (O R exons) that are alternatively spliced between said two samples.

12. The method according to claim 11 further wherein said extracted exon signal estimates are obtained by a method comprising:
extracting total RNA from said samples;
generating labeled cDNA targets from preparations of said samples;
performing hybridization, scanning, and extraction of exon signal estimates on two or more exon arrays for said first and second biologic samples; and
estimating the probability that each probeset was detected above background.

13. The method according to claim 12 further wherein:
said extraction of exon signals comprises normalizing data and generated signal estimates using Robust Multichip Analysis (RMA).

14. The method according to claim 11 further comprising:
correcting for multiple hypothesis testing using Benjamini-Hochberg method to reject falsely significant results.

15. The method according to claim 11 further comprising:
for the purpose of identifying overall diagnostic exon alternative splicing, comparing a set of differently prepared first samples to a set of differently prepared second samples.

16. The method according to claim 15 further comprising:
for the purpose of identifying overall diagnostic exon alternative splicing, comparing a set of differently prepared first samples comprising pluripotent embryonic stem cell (ESC), such as Cyt-ES and HUES6-ES, to a set of differently prepared second samples of neural progenitor (NP) cells, such as Cyt-NP, HUES6-NP, and hCNS-SCns.

17. The method according to claim 11 wherein each of said extracted exon signal estimates comprise expression estimates of at least 1 million features, used to interrogate expression of at least 250,000 exon clusters.

18. The method according to claim 11 wherein each of said extracted exon signal estimates comprise expression estimates of at least 1 million exon clusters.

19. The method according to claim 11 wherein said sample materials include undifferentiated cells and differentiated cells.

20. The method according to claim 11 wherein said at least one technique to detect false-positive outliers comprises one or more selected from the group consisting of:
removing values whose Pearson correlation coefficient is less than a predetermined amount;
removing values that have a studentized residual greater than a specified amount.
removing values that have a leverage that is greater than a specified amount.

21. The method according to claim 11 further comprising:
selecting probesets wherein the log₂ signal estimate xᵢⱼ for probeset i in cell-type j satisfies two conditions: (i) 2<xᵢⱼ<10,000 for all conditions/cell-types j; and (ii) detection above background (DABG) p-value<0.05 for all replicates in at least one condition/cell type j.
selecting for robust regression analysis genes with five probesets that satisfy the two conditions above in order to be considered.

22. The method according to claim 11 further comprising:
perform robust regression method rlm with M-estimation and a maximum iteration setting of 30 to estimate the linear function yᵢ=αxᵢ+β;
for each probeset, compute an term eᵢ, which is the difference between the actual value yᵢ and the estimated value ξᵢ from the estimated function ξᵢ=Axᵢ+B, where A and B are estimates of α and β;
estimate error term variance by sₑ²=Σeᵢ²/(n=p), to estimate the variance of the predicted value, s_{ξi}²=sₑ²(n⁻¹+(xᵢ−μₓ)²/sₓ²(n−1)), where n referred to the number of points generated for each gene and p referred to the number of independent variables (e.g., p=2 in an example method); and μₓ=Σxᵢ²/n; sₓ²=n⁻¹Σ(xᵢ−μₓ)².

23. The method according to claim 20 further comprising:
perform robust regression method rlm with M-estimation and a maximum iteration setting of 30 to estimate the linear function yᵢ=αxᵢ+β;
for each probeset, compute an term e₁, which is the difference between the actual value yᵢ and the estimated value ξᵢ from the estimated function ξᵢ=Axᵢ+B, where A and B are estimates of α and β;
estimate error term variance by sₑ²=Σeᵢ²/(n−p), to estimate the variance of the predicted value, s_{ξi}²=sₑ²(n⁻¹+(xᵢ−μₓ)²/sₓ²(n−1)), where n referred to the number of points generated for each gene and p referred to the number of independent variables (e.g., p=2 in an example method); and μₓ=Σxᵢ²/n; sₓ²=n⁻¹Σ(xᵢ−μₓ)²; define the leverage hᵢ of the i^{th} point as hᵢ=n⁻¹+(xᵢ−μₓ)²/sₓ²(n−1)), where a point has a high leverage if hᵢ>3p/n.
calculate the covariance ratio, covᵢ=(sᵢ²/sᵣ²)^{P}/(1−hᵢ), which is the ratio of the determinant of the covariance matrix after deleting the i^{th} observation to the determinant of the covariance matrix with the entire sample and considered a point to have high influence if |covᵢ−1|>3p/n.
compute the studentized residuals, rstudentᵢ=eᵢ²/(s₍ᵢ₎²(1−hᵢ)^{0.5}), where s₍ᵢ₎2=(n−p)s_{c}²/(n−p−1)−eᵢ²/(n−p−1)(1−hᵢ), the error term variance after deleting the i^{th} point. As rstudentᵢ was distributed as Student's t-distribution with n−p−1 degrees of freedom, each rstudentᵢ value was associated with a p-value;
label a point to be an “outlier” if p<0.01.

24. A method of determining whether a cellular sample is a pluripotent stem cell or multipotent neural progenitor cell, the method comprising one or more of:
detecting the presence or relative isoform ratio of an alternative splicing isoform for EHBP1SLK;
RAI14;
CTTN;
SORBS1;
UNC84A; SIRT1;
MLLT10; or
POT1.

25. The method according to claim 24 further comprising:
for one or more of said genes, detecting the presence of a larger (exon-included) isoform or a smaller (exon-skipped) isoform.

26. A method of determining the differentiation state of a cell, the method comprising:
detecting the relative ratios of alternative splicing isoforms of one or more genes or exon sets, and correlating isoform ratios to differentiation.

27. The method according to claim 26 further wherein said isoform ratios are internally controlled.

28. The method according to claim 26 further wherein said isoform ratios are not sensitive, during isoform detection, to filtering and image quality.

29. The method according to claim 26 further wherein said alternative exons comprise one or more exons from one or more genes selected from the group consisting of:
EHBP1;
SLK;
RAI14;
CTTN;
SORBS1;
UNC84A;
SIRT1;
MLLT10;
POT1.

30. A method of locating an AS region be detecting a sequence motif associated with AS regions.

31. The method according to claim 30 wherein said motif is selected from the group listed on Table 1.

32. A computer readable medium containing computer interpretable instructions that when loaded into an appropriately configured information processing device will cause the device to operate in accordance with the method of claim 1.

33. A system for analyzing and detecting alternative splice (AS) exons between two biologic samples comprising:
an interface for receiving exon expression data from two different sample materials of at least one exon set of interest;
said exon expression data comprising expression values of at least three different exons;
a logic processor performing robust regression analysis of said exon expression data;
wherein said robust regression analysis determines a linearized regression while reducing an impact of any outliers to said linearized regression;
said processor detecting said outliers;
said processor analyzing said outliers to detect false-positive outliers; and
said processor outputting indications of said outliers that are not false positive outliers, said indications identifying one or more exons that are alternatively spliced between said two samples.

34. The system of claim 33 wherein said samples are from different cellular developmental stages.

35. The system of claim 33 wherein said samples include undifferentiated cells and differentiated cells.

36. The system of claim 33 wherein said exon expression data is data captured from exon expression arrays.

37. The system of claim 33 wherein said exon expression data is data read from one more sequence libraries.

38. The system of claim 33 wherein said exon expression data is data determined by sequencing and/or hybridization of DNA and/or RNA.

39. The system of claim 33 further comprising:
said processor normalizing said exon expression data prior said robust regression.

40. The system of claim 33 wherein said analyzing said outliers to detect false-positive outliers comprises one or more selected from the group consisting of:
removing values whose Pearson correlation coefficient is less than a predetermined amount;
removing values that have a studentized residual greater than a specified amount; and
removing values that have a leverage that is greater than a specified amount.

41. The system of claim 33 wherein:
said two biologic samples comprise pluripotent human embryonic stem cells (hESCs) and multipotent neural progenitor cells (NPs); and
said outliers that are not false positive outliers identify exon expressions that are able to predictively distinguish between pluripotent human embryonic stem cells (hESCs) and multipotent neural progenitor cells (NPs).

42. A system able to determine post RNA-transcription events (e.g., alternative splicing (AS) events or RNA degradation) that are different between first and second biologic samples comprising:
a logic processor with one or more logic modules comprising:
a data receiving module receiving extracted exon signal estimates from at least two biologic samples indicating exon presence and/or expression for a very large exon data set;
one or more gene models;
an estimator module computing gene-level estimates from said extracted exon signal estimates for said gene models and for a gene, determining a t-statistic and a corresponding p-value representing relative enrichment of expression of said gene between said first sample versus said second sample;
a selector module applying a p-value cutoff to identify enriched genes and for enriched genes, selecting probesets that (i) comprised three or more individual probes; (ii) were localized within the exons of said gene models; and (iii) were detected above background in at least one of the samples lines;
an analysis module performing a robust regression analysis of said selected probesets to determine if some probesets behaved unexpectedly between said first sample and said second sample to identify AS exons;
wherein said robust regression analysis determines a linearized regression while reducing an impact of any outliers to said linearized regression;
an outlier detecting module;
a false-positive detection module; and
an interface module for outputting indications of said outliers that are not false positive outliers, said indications identifying one or more probesets (O R exons) that are alternatively spliced between said two samples.
